(19) United States
(12) Patent Application Publication
(10) Pub. No.: US 2015/0118729 A1
(43) Pub. Date: Apr. 30, 2015

(54) CELL PROGRAMING VIA GEOMETRIC CUES

(71) Applicant: The Board of Trustees of the University of Illinois

(72) Inventors: Kristopher A. Kilian, Savoy, IL (US); Junmin Lee, Urbana, IL (US); Yanfen Li, Urbana, IL (US)

(21) Appl. No.: 14/527,367

(22) Filed: Oct. 29, 2014

Related U.S. Application Data
(60) Provisional application No. 61/897,178, filed on Oct. 29, 2013.

(51) Int. Cl. C12N 5/00

(52) U.S. Cl. CPC C12N 5/0068; C12N 2535/10; C12N 2533/12; C12N 2533/52; C12N 2533/54
USPC 435/176; 435/402

(57) ABSTRACT

Disclosed herein are compositions and methods for programming a cell. The compositions include a substrate and a cell adhesion agent. The substrate includes a surface having a micropatterned object and the cell adhesion agent is immobilized within a first area defined by the micropatterned object.

### CROSS-REFERENCE TO RELATED APPLICATIONS

This application claims benefit of priority under 35 U.S.C. 119 to U.S. provisional patent application Ser. No. 61/897,178, filed Oct. 29, 2013, and entitled “PROGRAMING VIA GEOMETRIC CUES,” the contents of which are herein incorporated by reference in its entirety.

### STATEMENT REGARDING FEDERALLY SPONSORED RESEARCH OR DEVELOPMENT

This invention was made with government support under HL121757 awarded by the National Institutes of Health. The United States Government has certain rights in the invention.

### FIELD OF INVENTION

The present disclosure relates to compositions, substrates and methods for cell programming. In particular, microengineered cell culture materials and methods are disclosed that provide for cellular programming.

### BACKGROUND

Yamanaka and colleagues (2012 Nobel Prize in Physiology and Medicine) and the Thompson laboratory have demonstrated that somatic cells can be reprogrammed to earlier developmental states by ectopic expression of specific transcription factors. These induced pluripotent stem cells (“iPSC”) are attractive for regenerative therapies as patient-specific cells may be reprogrammed, differentiated or genetically modified to enable autologous regeneration. However, stochastic processes govern most reprogramming methods that result in long times (several weeks to months), low efficiency, and the use of oncogenes through viral transduction methods.

To improve on the reprogramming process, there has been an intense research effort into the de-differentiation mechanism and the search for better, more clinically viable methods. At the heart of current research efforts is a need to 1) improve the efficiency of the reprogramming process—which typically remains at <1% after 4 weeks—and 2) develop strategies that eliminate the need for viral vectors that pose a risk of genome integration. Towards this end, workers in the field have made improvements to the reprogramming procedure via a number of methods including the use of multipotent cells, alternative genetic techniques, recombinant proteins, mRNA, microRNAs, and small molecules that target chromatin modifying enzymes and other signaling pathways. While there have been numerous advances in optimizing and understanding the reprogramming process, little is known about the cues that initiate these rare reprogramming events.

The current view of somatic cell reprogramming separates the process into multiple distinct phases: the initiation phase, an intermediate phase—postulated to be rate-limiting—that is not currently understood, and a late phase that includes maturation and stabilization Initially, viruses encoding pluripotency factors are introduced to the somatic cells in culture that initiates the ‘stochastic’ phase where histones are modified at somatic genes, proliferation increases and a small subset of cells begins to undergo a mesenchymal-to-epithelial transition (“MET”). Next, it is postulated that there exists a rate limiting intermediate phase where endogenous pluripotency programs are activated. Finally, the deterministic (hierarchical) phase leads to the final establishment of pluripotency regulatory networks and resetting of the cellular epigenetic state (FIG. 1). Current reprogramming methods rely on tissue culture plastic that leads to broad heterogeneity in cell density, packing and morphology across the substrate (FIG. 2A).

### SUMMARY

In a first respect, a composition for programming a cell is disclosed. The composition includes a substrate and a cell adhesion agent. The substrate includes a surface having a micropatterned object and the cell adhesion agent is immobilized within a first area defined by the micropatterned object.

In a second respect, a method for programming a cell is disclosed. The method includes three steps. The first step is providing a composition for programming the cell. The composition includes a substrate and a cell adhesion agent. The substrate includes a surface having a micropatterned object and the cell adhesion agent is immobilized within a first area defined by the micropatterned object. The second step is contacting a cell with the composition to form an adherent cell culture. The third step is culturing the adherent cell culture for a period to effect programming of the cell.

In a third aspect, a kit for programming a cell is disclosed. The kit includes a premade composition for programming a cell. The premade composition includes a substrate and cell adhesion agent. The substrate includes a surface having a micropatterned object and the cell adhesion agent is immobilized within a first area defined by the micropatterned object.

These and other features, objects and advantages of the present invention will become better understood from the description that follows. In the description, reference is made to the accompanying drawings, which form a part hereof and in which there is shown by way of illustration, not limitation, embodiments of the invention.

### BRIEF DESCRIPTION OF THE DRAWINGS

FIG. 1 depicts a scheme showing a prior art understanding of the phases of reprogramming cells.

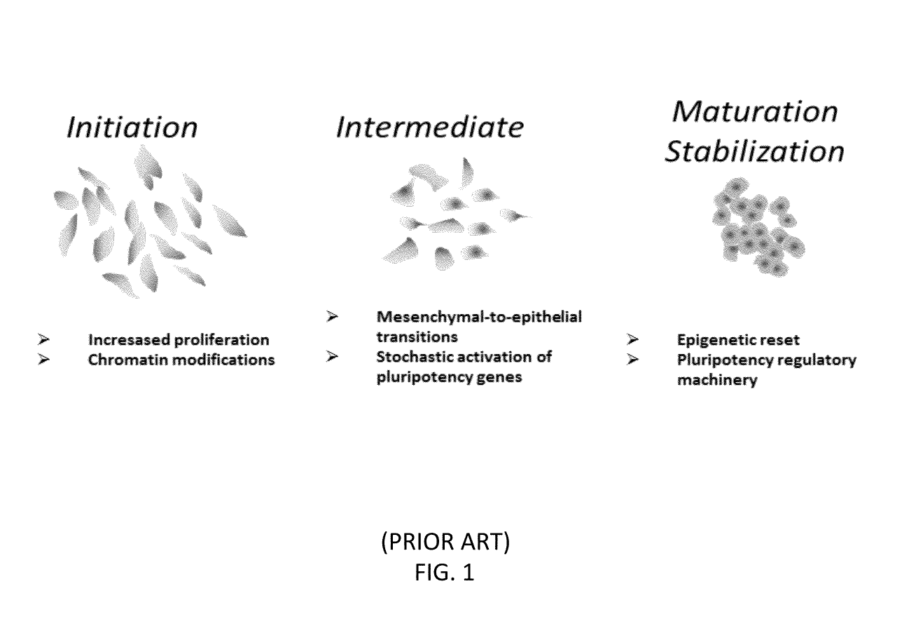

FIG. 2A depicts an illustration of a tissue culture plate having an unpatterned microsurface (inset (i)) that provides cell reprogramming according to prior art technologies (inset (ii)). Cellular key: MEF, mouse embryonic fibroblasts; pre-iPSC, pre-induced pluripotent stem cells; and iPSC, induced pluripotent stem cells.

FIG. 2B depicts for one embodiment of a tissue culture plate having a patterned microsurface (inset (i)) for forcing cell morphological changes to induce cell programming more efficiently (inset (ii)). Cellular key as in FIG. 2A.

FIG. 3A depicts a schematic showing an exemplary first chemistry and exemplary second chemistry for patterning a substrate surface, wherein moieties X and Y denote functional groups that couple the first and second chemistries to the substrate surface and the moieties W and Z denote functional groups that couple the first or second chemistries to the desired cell adhesion agent.

FIG. 3B shows a schematic showing an exemplary chemistry used to pattern cell adhesion o a gold substrate surface.

FIG. 3C depicts one preferred embodiment for making micropatterned substrates of FIG. 3A for cell culture substrates.

FIG. 3D depicts a chemical scheme for conjugating matrix proteins to hydrogels of variable stiffness.

FIG. 4A shows multipotency marker enhancement (Stro-1) after patterning; inset (i) shows patterned cells; inset (ii) shows non-patterned cells; inset (iii) shows quantitation of multipotency marker for insets (i) and (ii); scale bar, 200 μm for insets (i) and (ii). The double asterisks refer to statistically significant differences in the data presented in inset (iii).

FIG. 4B shows multipotency marker enhancement (Endoglin) after patterning; inset (i) shows patterned cells; inset (ii) shows non-patterned cells; inset (iii) shows quantitation of multipotency marker for insets (i) and (ii); scale bar, 200 μm for insets (i) and (ii). The double asterisks have the same meaning as in FIG. 4A.

FIG. 5A depicts exemplary geometries and shapes to alter the effects of curvature and pinch points in guiding proliferation, chromatin architecture and METs, wherein 1: circle; 2: square; 3: triangle; 4: square-edged cross shape; 5: starburst shape; 6: flower shape; 7: round-edged cross shape; 8: beveled-edged cross shape; 9: spiral shape; 10: “H” type 1 shape; 11: “H” type 2 shape; 12: “H” type 3 shape; 13: 5-point star, and 14: 8-point snowflake shape are shown.

FIG. 5B depicts a finite element analysis (contractile cell monolayers modeled with ABAQUS software) used to select a range of geometries that present variable regions of tension and compression on a large population of cells. The objects are denoted as in FIG. 5A.

FIG. 5C depicts the results of mouse embryonic fibroblasts seeded on substrates micropatterned with circle geometries.

FIG. 5D depicts the results of embryonic fibroblasts seeded on substrates micropatterned with square geometries; scale bar, 200 μm.

FIG. 5E depicts the results of embryonic fibroblasts seeded on substrates micropatterned with cross geometries.

FIG. 6A illustrates regions of highly proliferative clusters of cells that stain positive with alkaline phosphatase.

FIG. 6B illustrates regions of concavity at the interstices of a shape approximating a flower-shaped geometry promotes METs.

FIG. 6C depicts immunostaining of mouse embryonic fibroblasts show elevated staining for histone marks (H3K9ac [sub-panel ((ii)] and H3K4me2 [sub-panel ((iii)]) involved in programming at the perimeter of flower-shaped geometries. Sub-panel (i) depicts immunostaining of mouse embryonic fibroblasts for acetyl-K marker. Sub-panels (iv), (v) and (vi) are immunostaining controls for sub-panels (i), (ii) and respectively.

FIG. 7A illustrates in sub-panel (i) a micropatterned substrate geometry upon which cells were seeded wherein regions of pinch-points (1), concavity (2, 3) and unpatterned controls (4) and in the boxed region (sub-panel (ii)) an enhanced immunofluorescence of lysine acetylation in regions of concavity (2, 3) and pinch-points (1) promote enhanced lysine acetylation compared to central regions and unpatterned control cultures of MEFs.

FIG. 7B depicts a quantitation of immunofluorescence of lysine acetylation in the regions of pinch-points (1), concavity (2, 3) and unpatterned controls (4) depicted in FIG. 7A.

FIG. 8 depicts an exemplary embodiment whereby cascading geometric patterns can be used to modulate degrees of curvature across the micropatterned substrate surface.

FIG. 9A illustrates phase contrast images of MEFs adherent to unpatterned gels (panel inset (i)) and micropatterned gels (panel inset (ii)) with different stiffness at different time points.

FIG. 9B illustrates immunofluorescence images of MEFs patterned on the 1 kPa hydrogel (sub-panel (i)) show higher expression and cytoplasmic localization of E-cadherin compared to 10 kPa hydrogel (sub-panel (ii)) and 100 kPa hydrogel (sub-panel (iii)); control glass substrate is shown in sub-panel (iv).

FIG. 10A depicts an un-patterned MEF population exposed to lentivirus encoding OSKM at day 1 in culture.

FIG. 10B depicts a patterned MEF population exposed to lentivirus encoding OSKM at day 1 in culture.

FIG. 10C depicts an un-patterned MEF population exposed to lentivirus encoding OSKM at day 11 in culture.

FIG. 10D depicts a patterned MEF population exposed to lentivirus encoding OSKM at day 11 in culture.

FIG. 10E depicts a quantitation of colony size at day 11 in culture for the un-patterned population (control) and patterned population (“μCP”).

FIG. 10F depicts a quantitation of colony number at day 11 in culture for the unpatterned population (control) patterned population (“μCP”).

FIG. 10G depicts RT-PCR analysis of endogenous pluripotency marker expression for iPS cells and MEFs.

FIG. 11A depicts iPSCs generated on patterned surfaces express endogenous Oct4.

FIG. 11B depicts iPSCs generated on patterned surfaces stain positive for alkaline phosphatase.

FIG. 12A depicts soft hydrogels promote expression of pluripotency markers, wherein MEFs were cultured for 1 week in 10,000 μm² patterns on 1 kPa fibronectin modified polyacrylamide (inset (i)) compared to those cultured on fibronectin modified glass (inset (ii)).

FIG. 12B depicts quantitation of the average number of cells captured to a given patterned surface of the specified stiffness.

FIG. 12C depicts the percentage of programmed cells on a given patterned surface of the specified stiffness.

FIG. 13A depicts exemplary heat maps comparing the expression of E-cadherin as a function of substrate mechanical properties and composition of matrix protein as cell adhesion agent (F, fibronectin; L, laminin; C, collagen; or combinations thereof (FLC)).

FIG. 13B depicts exemplary heat maps comparing the expression of Oct-4 as a function of substrate mechanical properties and composition of matrix protein as cell adhesion agent (F, fibronectin; L, laminin; C, collagen; or combinations thereof (FLC)).

FIG. 13C depicts DAPI staining (sub-panels (i), (v) and (ix)), E-cadherin immunostaining (sub-panels (ii), (vi) and (x)), Oct4 immunostaining (sub-panels (iii), (vii) and (xi)) and merged staining/immunostaining patterns (sub-panels (iv), (viii) and (xii)) for cells anchored by laminin (sub-panels (i),-(iv)), or by a combination of fibronectin, laminin and collagen (sub-panels (v)-(viii)), in comparison to control glass substrates incubated in the presence of a combination of fibronectin, laminin and collagen (sub-panels (ix)-(xii)).

FIG. 14 depicts programming of B16 cells seeded in different sized patterns of 1,000 μm² (“1,000”), 3,000 μm² (“3,000”), 5,000 μm² (“5,000”), 20,000 μm² (“20,000”) and 100,000 μm² (“100,000”) on polyacrylamide hydrogels having different stiffness, and immunostained for expression of cellular marker ABCB5. NP, non-patterned; G, glass substrate alone.

FIG. 15 depicts immunostaining of cells programmed on different geometries composed of circle shape patterns (sub-panels (i)-(vi)); round-edged cross shape patterns ((sub-panels (vii)-(xii)); beveled-edged cross shape patterns (sub-panels (xiii)-(xviii)); flower shape patterns (sub-panels (xix)-(xxiv)); starburst shape patterns (sub-panels (xxv)-(xxx)); triangle shape patterns (sub-panels (xxxi)-(xxxvi)); t square shape patterns (sub-panels (xxxvii)-(xlii)); and spiral shape patterns (sub-panels (xliii)-(xlviii)) for simulation (sub-panels (i), (vii), (xiii), (xix), (xxv), (xxxi), (xxxvii), and (xliii)); ABCB5 (sub-panels (ii), (viii), (xiv), (xx), (xxvi), (xxxii), (xxxviii), and (xliv)); CD271 (sub-panels (iii), (ix), (xv), (xxi), (xxvii), (xxxiii), (xxxix), and (xlv)); CD133 (sub-panels (iv), (x), (xvi), (xxii), (xxviii), (xxxiv), (xl), and (xlvi)); NanOg (sub-panels (v), (xi), (xvii), (xxiii), (xxix), (xxxv), (xli), and (xlvii)); and Oct4 (sub-panels (vi), (xii), (xviii), (xxiv), (xxx), (xxxvi), (xlii), and (xlviii)).

FIG. 16 depicts an exemplary heat map of a marker panel for murine melanoma cell lines, B16F0 and B16F10 seeded on glass, hydrogel or spiral shaped patterned hydrogels.

FIG. 17A depicts a strategy for programming cancer cells to stem cells using geometric patterning and inhibitors of MAP kinases.

FIG. 17B depicts an exemplary immunostaining of cancer cell marker CD271 for cell cultures grown on glass substrates (sub-panels (i)-(iv)) or circle shaped patterned substrates (sub-panels (v)-(viii)) for cells treated with no MAP kinase inhibitors (sub-panels (i) and (v)), or with MAP kinase inhibitor SB202190 against the p38 protein (sub-panels (ii) and (vi)), with MAP kinase inhibitor SP600125 against the JNK protein (sub-panels (iii) and (vii)) or with the MAP kinase inhibitor FR180204 against the ERK1/2 (“ERK”) protein (sub-panels (iv) and (viii)).

FIG. 17C depicts an exemplary relative staining pattern intensities of CD271 marker expression for cells seeded on different shaped geometry pattern or on control glass substrates (“G”) and cultured in the absence (“CTR”) or presence of MAP kinase inhibitors, SB202190 (p38), SP600125 (JNK), and FR180204 (ERK1/2).

FIG. 17D depicts an exemplary heat map of marker gene expression for cells seeded on a control glass substrate, a hydrogel substrate or a spiral-shaped patterned substrate.

### DETAILED DESCRIPTION

The present disclosure provides details of the discovery of novel solutions to the technological barriers of low efficiency in cellular programming. These technological bathers are attributed in part to the physical and biochemical attributes of the cell culture substrate during the early initiation phase. Similar to how small molecules that target chromatin modifying enzymes have been shown to increase programming efficiency, patterned substrates presenting geometric cues and tunable stiffness are disclosed that guide cellular morphology that influence the epigenetic state of adherent cells to drive somatic cell programming. The present disclosure provides examples that demonstrate microengineered geometric cues promote transitions between epithelial and mesenchymal phenotypes and between metastatic cancer cell and cancer stem cell phenotypes and the ability to control the acetylation state of chromatin and gene expression in micropatterned cells by utilizing patterned substrates to control the underlying cell geometry.

The new materials disclosed herein lower the epigenetic bather to somatic cell programming. The disclosed improvements to current methods afford new applications of the de-differentiation process of significance to the field. Guiding programming through substrate properties alone is a new direction in programming that does not involve the delivery of viral vectors or animal derived products. Since most current methods involve components that are unacceptable for use in humans, the disclosed materials and methods are more amenable to clinical translation. These engineered substrates provide tools to direct programming phases (for instance, by using geometry to force MET). Finally, engineered substrates can be fabricated to reduce programming time, increase programming efficiency and replace one or more viral vectors.

These materials will allow better control over cell adhesion and proliferation to systematically study the phases of programming. Moreover, the disclosed technology is amenable to programming somatic cells regardless of phenotype. Thus, the materials and methods disclosed herein can be adapted to program cancer cells to cells having a pluripotent cancer stem cell phenotype. Such programming events provide valuable targets for preventing cancer recurrence. Furthermore, materials and tools disclosed herein have broad applicability in research laboratories and clinical settings in the realms of regenerative medicine and cancer biology.

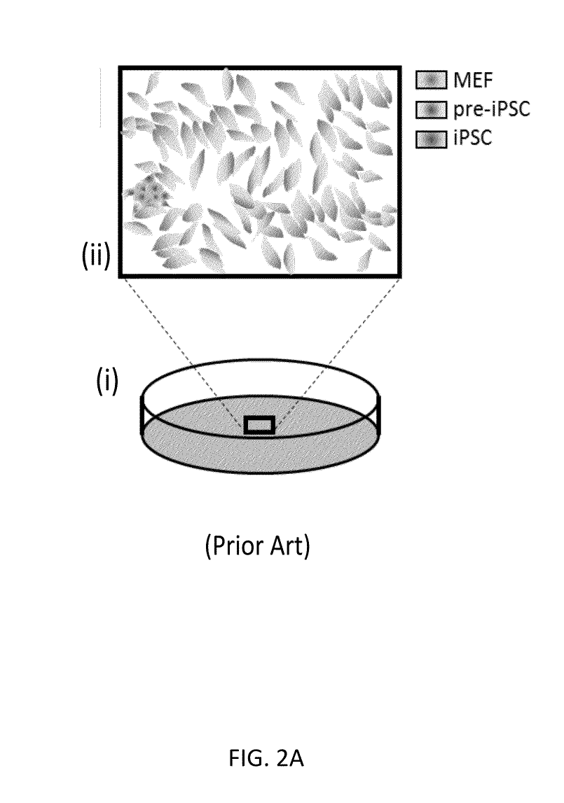

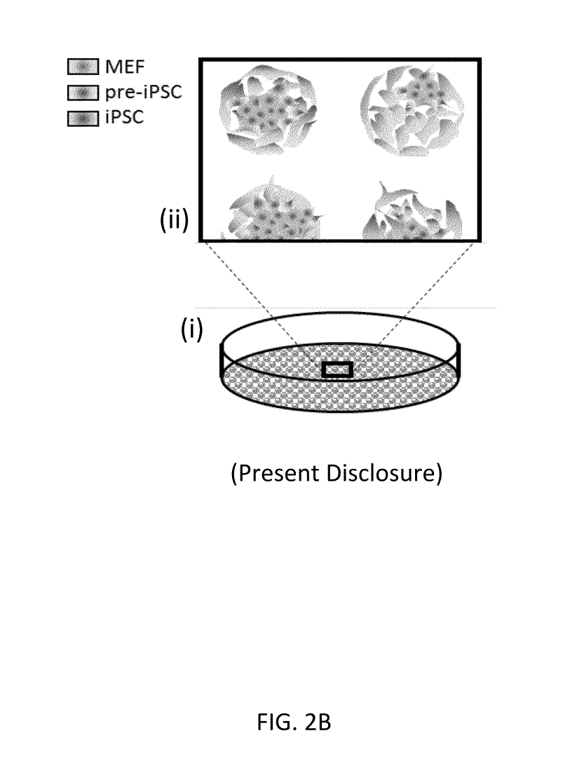

Using designer substrates that modulate geometry and substrate mechanics is a novel method to study and direct the early morphological events that precede programming (for example, MET, chromatin de-condensation, etc.; see FIG. 2B). Recent work from the inventors provides a rationale basis for how controlling stem cell geometry can modulate nuclear architecture and gene expression. Similarly, geometric cues can be used to guide cellular morphogenesis and lower the epigenetic barrier to programming. This approach is different from conventional strategies because it uses microengineering tools to study how physical cues that coordinate cell packing and morphology will influence somatic cell programming. Improved control over cell morphology and proliferation characteristics will augment fundamental studies aimed at elucidating the cues that initiate programming. Furthermore, since programming events are rare using current methods, the disclosed strategy will significantly decrease time and improve efficiency, and potentially obviate the need for exogenous delivery of viral factors.

### TERMINOLOGY AND DEFINITIONS

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

Terms used herein are intended as “open” terms (e.g., the term “including” should be interpreted as “including but not limited to,” the term “having” should be interpreted as “having at least,” the term “includes” should be interpreted as “includes but is not limited to,” etc.).

The phrase “such as” should be interpreted as “for example, including.”

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The term “programming” refers to changing the phenotype and/or function of a cell from a first state to a second state. First and second states include the following non-limiting examples: first state: a stem or progenitor cell and second state: a differentiated cell; first state: a differentiated cell and second state: a de-differentiated cell of earlier developmental state; first state: a differentiated cell and second state: a trans-differentiated cell belonging to a different germ layer.

The term “coupled” refers to binding a first entity to a second entity by known chemistry, such as covalent bonding, ionic bonding, Van der Waals forces, hydrogen bonding and metallic bonding.

### Compositions and Fabrication Methods for Micro-Patterned Substrates Having Geometric Cues

In a first aspect, a composition for programming a cell is disclosed. The composition includes a substrate and a cell adhesion agent. The substrate includes a surface having a variety of compositions. Exemplary surface compositions include a metal, such as gold; glass; glass-polydimethylsiloxane (PDMS) composites; polyacrylate; polystyrene; polycarbonate; graphene-ITO glass composites; or hydrogels, such as chemically functionalized polyacrylamide gels.

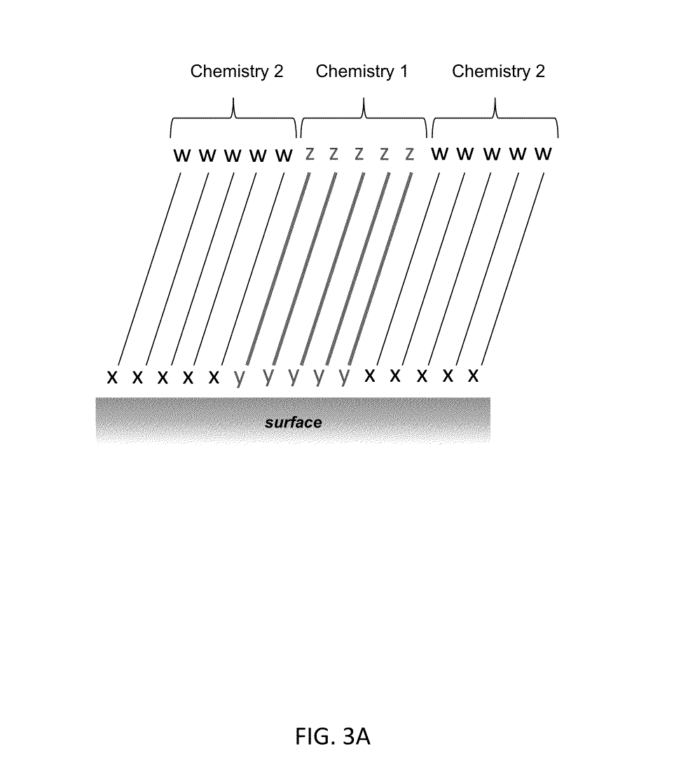

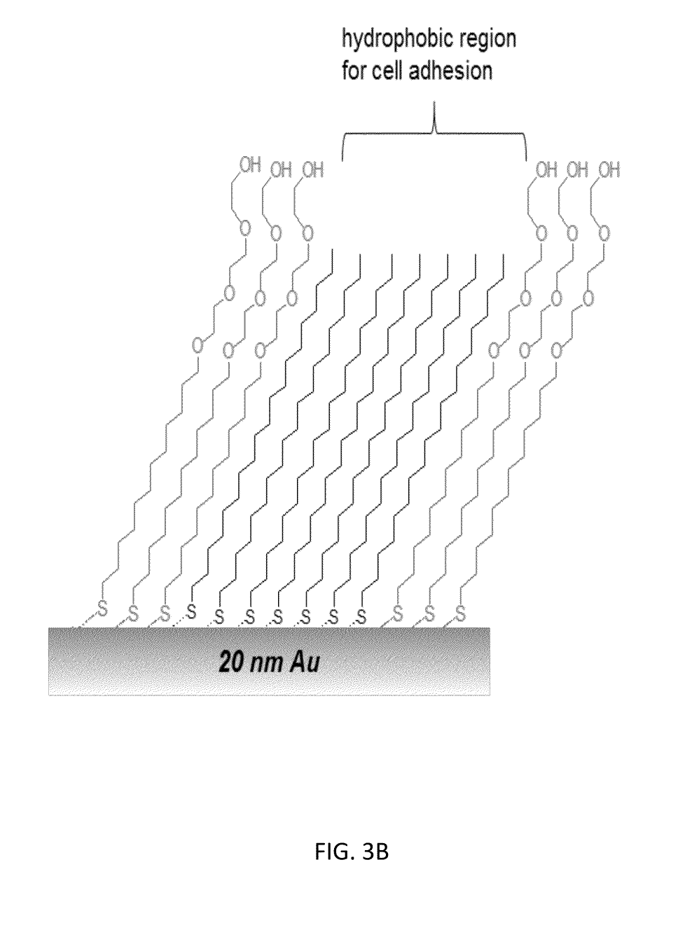

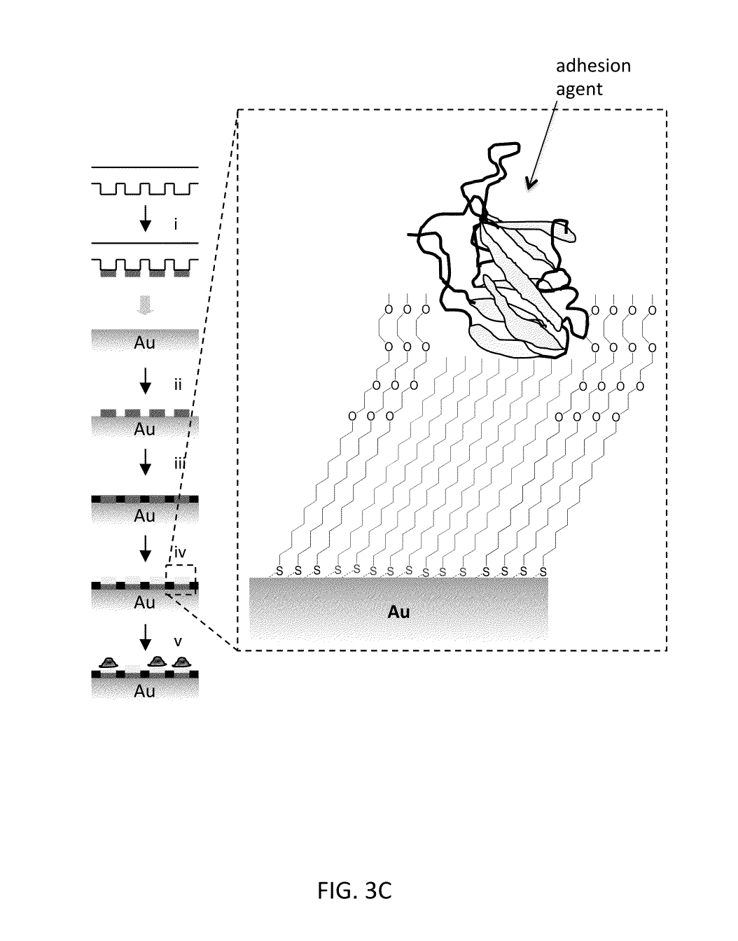

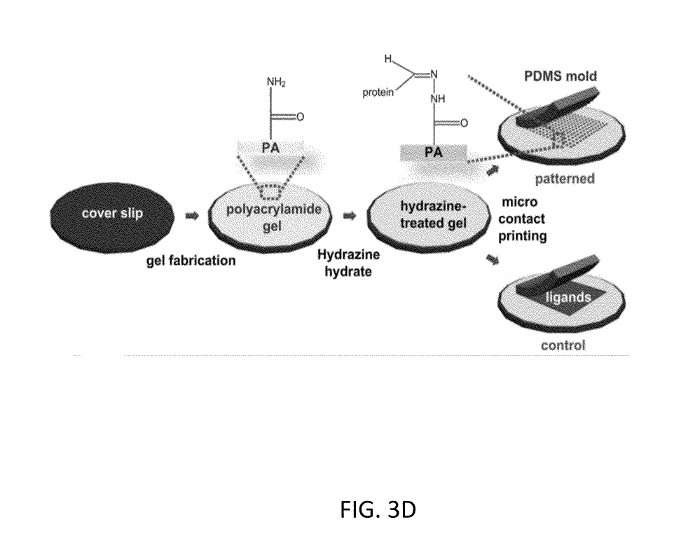

Referring to FIG. 3A, the substrate includes a surface having a micropatterned object. The micropatterned object includes a first area having one of a first chemistry or a second chemistry attached to the substrate surface. The remainder of the substrate surface defines a second area having one of a first chemistry or a second chemistry attached to the substrate surface. The first and second areas of the substrate surface have different chemistries. In the case of the aforementioned first and second chemistries, the first and second areas of the substrate surface have different first and second chemistries.

One of the first chemistry or the second chemistry include terminal group functionality to couple the substrate surface to the cell adhesion agent. In some aspects, the cell adhesion agent is immobilized in the first area defined by an inner boundary of the micropatterned object. In other aspects, the cell adhesion agent is immobilized within the second area defined by an outer boundary of the micropatterned object.

In some embodiments, surface assembled monolayers (SAMs) of alkanethiols on gold can be used to control the surface properties. By using alkanethiols terminated with a ligand for a specific molecule or an end group for a specific chemistry (for example, “click” chemistry), the surfaces of SAMs can be modified to incorporate specified ligands. The alkanethiols described herein can have a length in the range from C₄-C₃₀, where alkanethiols having a length of C₁₈ are preferred. Microcontact printing using soft PDMS stamps can then be used to pattern the alkanethiols into the desired pattern as a first chemistry and passivate the outside of the pattern using PEG-terminated alkanethiols as a second chemistry (see FIGS. 3B, C). The desired cell adhesion agent can then be conjugated to the alkanethiol using the desired chemistry. See, for example, K. A. Kilian, B Bugarija, B. T. Lahn and M. Mrksich, “Geometric cues for directing the differentiation of mesenchymal stem cells,” Proc. Nat'l. Acad. Sci., U.S.A. 107:4872-4877 (2010).

In other embodiments, SAMs can be precisely localized on the substrate surface. In this application, an inert alkanethiol SAM is formed on a gold substrate surface. A microfluidic device can be used to run an aqueous solution over the desired areas to locally remove regions of this bioinert alkanethiol as a first chemistry and then another ‘active’ alkanethiol is run through the microchannels to place a SAM with the desired chemical properties as a second chemistry to couple to the desired cell adhesion agent. See, for example, J. T. Koepsel and W. L. Murphy, “Patterning Discrete Stem Cell Culture Environments via Localized Self-Assembled Monolayer Replacement,” Langmuir 25:12825-12834 (2009).

In other embodiments, an elastomeric membrane patterning of substrate surfaces can be used. A perforated thin PDMS membrane can be formed via photolithography or another process where the holes in the membrane correspond to the desired patterns. The membrane is placed over the desired substrate, such as a glass or polymer substrate (for example, polystyrene or polyacrylate) and the required protein is added to the surface as a first chemistry. BSA can be added after removal of the membrane to passivate the outside of the patterns as a second chemistry. See, for example, E. Ostuni, R. Kane, C. S. Chen, D. E. Ingber and G. M. Whitesides, “Patterning Mammalian Cells Using Elastomeric Membranes,” Langmuir 16:7811-7819 (2000). For an example of this technique on polyacrylamide hydrogel substrates using Sulfo-SANPAH chemistry, see Wang, N., Ostuni, E., Whitesides, G. M. and Ingber, D. E. “Micropatterning tractional forces in living cells,” Cell Motil. Cytoskeleton 52: 97-106. doi: 10.1002/cm.10037 (2002).

In other embodiments, silane-based patterning of substrate surfaces can be used. In this approach, multiple silane-based chemistries can be used to functionalize silicon oxide and glass substrates for patterning. One can also form SAMs on these substrate surfaces. The surfaces can be patterned through microcontact printing or other methods such as photolithographic degradation of the silane layer. See, for example, C. Hoffmann, G. E. M. Tovar, “Mixed self-assembled monolayers (SAMs) consisting of methoxy-tri(ethylene glycol)-terminated and alkyl-terminated dimethylchlorosilanes control the non-specific adsorption of proteins at oxidic surfaces,” J. Coll. Inter. Sci., 295:427-435 (2006).

In other embodiments, micropatterning with pluronics can be used as substrate surfaces. In this approach, pluronics (for example, PEO-PPO-PEO) can be applied to glass/PDMS surfaces using microfluidics or lithographic techniques as a first chemistry. The pluronics provide areas of nonadhesiveness on the substrate surface, wherein the cell adhesion agent or cells cannot bind. The remaining area of the substrate surface that includes glass/PDMS surfaces having no pluronics layer can be used as the second chemistry to couple to the desired cell adhesion agents. See, for example, V. A. Liu, W. E. Jastromb, S. N. Bhatia, “Engineering protein and cell adhesivity using PEO-terminated triblock polymers,” J. Biomed. Mater. Res. 60:126-34 (2002).

In other embodiments, graphene can be used as substrate surfaces. In this approach, a reduced graphene oxide layer is formed on the surface of ITO glass. Photoresist is coated on top and then excess graphene oxide (outside the pattern) is removed by plasma treatment to provide a first chemistry. PEDOT is then deposited electrochemically on ITO and the rest of the photoresist removed. The PEDOT surface can then be functionalized for passivation outside of the pattern as a second chemistry. See, for example, Hsiao, Y.-S., Kuo, C.-W. and Chen, P. “Multifunctional Graphene—PEDOT Microelectrodes for On-Chip Manipulation of Human Mesenchymal Stem Cells,” Adv. Funct. Mater. 23: 4649-4656 (2013), doi: 10.1002/adfm.201203631

In other embodiments, stamp-offs can be used as substrate surfaces. In this approach, a PDMS stamp is inked with a specific chemistry and stamped onto a UV oxidized template, and the template defines a first chemistry. This strategy allows multiple different chemistries to be patterned on a substrate. See for example, Desai, R. A., et al. “Subcellular spatial segregation of integrin subtypes by patterned multicomponent surfaces,” Integrative Biology 3: 560-567 (2011).

In other embodiments, microcontact printing on hydrazine hydrate activated polyacrylamide can be used as substrate surfaces. In this approach, and referring to FIG. 3D, polyacrylamide hydrogels are treated with hydrazine hydrate and acetic acid to convert amide groups of the polyacrylamide to active hydrazide groups as a first chemistry. The resultant active hydrazide groups can be reacted with cell adhesion agents having aldehydes. Exemplary cell adhesion agents include post-translationally modified proteins oxidized to convert side sugar groups into aldehyde groups. The resultant oxidized proteins can be stamped onto the surface of the activated polyacrylamide surface. See, for example, V. Damljanovic, B. C. Lagerholm, K. Jacobson, “Bulk and micropatterned conjugation of extracellular matrix proteins to characterized polyacrylamide substrates for cell mechanotransduction assays,” BioTechniques 39:847-851 2005).

In other embodiments, microcontact printing on Sulfo-SANPAH activated polyacrylamide can be used as substrate surfaces. In this approach, Sulfo-SANPAH is used to activate the polyacrylamide gel surface as a first chemistry. Proteins with amine groups can then be microcontact printed using PDMS stamps onto the gel surfaces. BSA may be used to passivate the outsides of the patterns as a second chemistry to decrease non-specific adhesion. See, for example, S. Chirasatitsin and A. J. Engler, “Detecting cell-adhesive sites in extracellular matrix using force spectroscopy mapping,” J. Phys. Condens. Matter. 22:194102 (2010).

In other embodiments, micropatterning using acrylic acid N-hydroxysuccinimide (NHS-Acrylate) can be used as substrate surfaces. In this approach, activated NHS-ester groups are incorporated into the prepolymer polyacrylamide solution. When the polymer is formed, the activated NHS groups are covalently attached to the hydrogel as a first chemistry. Microcontact printing using PDMS stamps then can be used to pattern the desired cell adhesion agent onto the substrate surface. See, for example, S. R. Polio, K. E. Rothenberg, D. Stamenović, and M. L. Smith, “A micropatterning and image processing approach to simplify measurement of cellular traction forces,” Acta Biomater. 8:82-88 (2012).

In other embodiments, micropatterning using deep UV activation of polyacrylamide can be used as substrate surfaces. In this approach, the gel is cast onto a photomask with the required patterns. After the gel is polymerized, it is exposed to deep UV light. The UV exposure through the mask activates these areas as a first chemistry for coupling the desired cell adhesion agent (for example, protein). The desired cell adhesion agent is then pooled onto the gel surface and can attach where the gel was exposed to UV. See, for example, Q. Tseng et al., “A new micropatterning method of soft substrates reveals that different tumorigenic signals can promote or reduce cell contraction levels,” Lab Chip, 11:2231 (2011).

In other embodiments, transfer of patterned protein from glass to surface of polyacrylamide can be used as substrate surfaces. In this approach, protein as a cell adhesion agent is patterned using microcontact printing on a glass substrate as a first chemistry. Polyacrylamide pre-polymer solution is added and sandwiched with a coverslip. After the gel is formed, it is detached with the protein patterns being incorporated on the surface of the polyacrylamide hydrogel. See, for example, X. Tang et al. “A novel technique for micro patterning proteins and cells on polyacrylamide gels,” Soft Matter, 8:7197-7206 (2012).

In other embodiments, PEG polymer brush based patterning onto glass can be used as substrate surfaces. In this approach, PEG-silane is deposited onto glass and then photoresist is spincoated onto it. Using lithography, the desired pattern is exposed to UV and plasma treated to remove the PEG silane. Initiator is added in its place as a first chemistry and the rest of the photoresist is stripped off to form the second chemistry. The polymer brushes are formed off the initiator to form the adhesive pattern. See, for example, Z. Zhou, P. Yu, H. M. Geller, and C. K. Ober, “Biomimetic Polymer Brushes Containing Tethered Acetylcholine Analogs for Protein and Hippocampal Neuronal Cell Patterning,” Biomacromolecules 14:529-537 (2013).

In other embodiments, micro-nanostructured polyethylene glycol diacrylate (PEG-DA) patterning onto gold can be used as substrate surfaces. In this approach, gold nanoparticle arrays are fabricated by block copolymer micellar nanolithography. A layer of photoresist is then applied to cover the array, and lithography is used to form a microstructure of the desired pattern. The excess gold particles are blown off then the rest of the photoresist is removed giving gold nanoparticles in an array in micro-sized shapes. PEGDA and initiator are cast together over this array, and a polymerized PEGDA hydrogel with patterns consisting of gold nanoparticles as a first chemistry forms thereafter. See, for example, D. Aydin et al., “Polymeric Substrates with Tunable Elasticity and Nanoscopically Controlled Biomolecule Presentation,” Langmuir 26:15472-15480 (2010).

In other embodiments, photoconjugation of pendant functionalities onto hydrogels can be used as substrate surfaces. In this approach, multiple click-based chemistries can be used that utilize photoconjugation. Sterolithography can be used to conjugate only moieties where a laser is pointed giving the ability to pattern ligands in complicated 3d manners within a PEG backbone as a first chemistry. The technique is flexible and can be utilized for other purposes such as photocleavage of moieties or photo-degradation of hydrogel. See, for example, DeForest, C. A., Anseth, K. S., “Cytocompatible click-based hydrogels with dynamically tunable properties through orthogonal photoconjugation and photocleavage reactions,” Nat. Chem. 23:925-31 (2011).

The first or second chemistries can include terminal functionalized groups tailored to couple to the desired substrate surfaces (as described supra) and to the desired cell adhesion agents. Preferred cell adhesion agents include proteins, peptides, lectins and polysaccharides. Such cell adhesion agents can derive from natural sources, recombinant sources, or synthetic sources. Cell adhesion agents can also be genetically or chemically modified to include complementary moieties for reactive coupling to the terminal functionalized groups of the first or second chemistries. Preferred proteins as cell adhesion agents include fibronectin, laminin, collagen and vitronectin, among others, including mixtures thereof.

Referring to FIG. 3C, the manner of constraining the geometry of stem cells can enhance multipotency and preserve “stemness.” Geometric cues have been identified that guide a MET using a self-assembled monolayer chemistry of alkanethiolates on gold to define hydrophobic regions across a substrate with an inert intervening background. Briefly, and with reference to FIG. 3C, thin layers of titanium (4 nm) followed by gold (20 nm) can be deposited by E-beam evaporation (Temescal) onto glass coverslips (FIG. 3C, final “Au” substrate). Silicon masters can then be generated by photolithography using a laser printed mask onto 20-30 μm thick layer of photoresist (SU-8, Microchem) and polydimethylsiloxane stamps are fabricated upon this template (FIG. 3C, t). Stamps are ‘inked’ briefly in a solution of octadecanthiol in ethanol (10 mM), dried under flow of nitrogen and brought into contact with the gold surface for two minutes (FIG. 3C, it). The surfaces are immersed in a solution of triethylene glycol undecanethiol (EG3) to render the regions around the stamped features inert (FIG. 3C, iii). Next, a suitable adhesion agent, such as a protein (for example, fibronectin (25 μg/mL in 1×PBS)), is applied to the surface to provide cell adhesive points (FIG. 3C, iv). Finally, the cells are seeded on the micropatterned substrate surface (FIG. 3C, v).

### Cell Programming on Micro-Patterned Substrates Having Geometric Cues

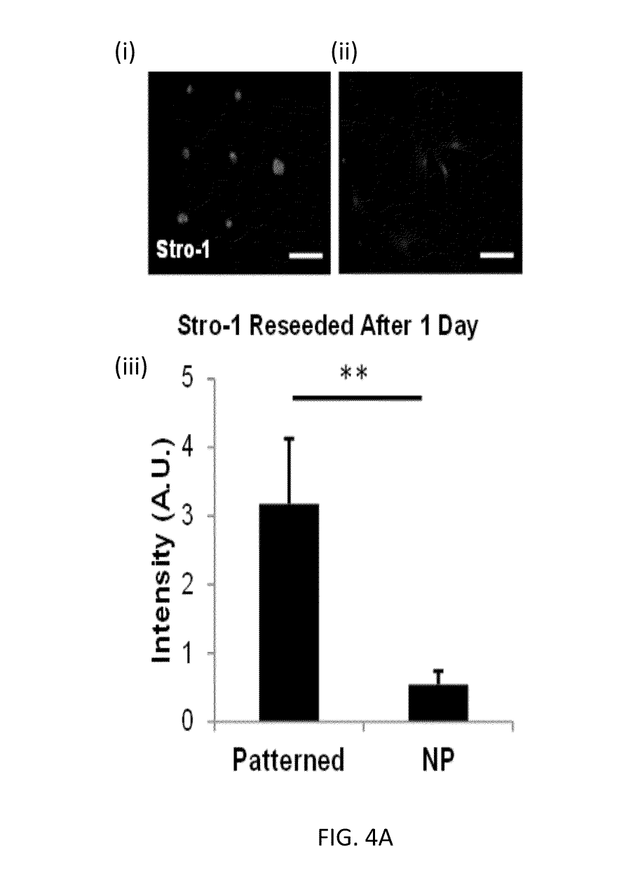

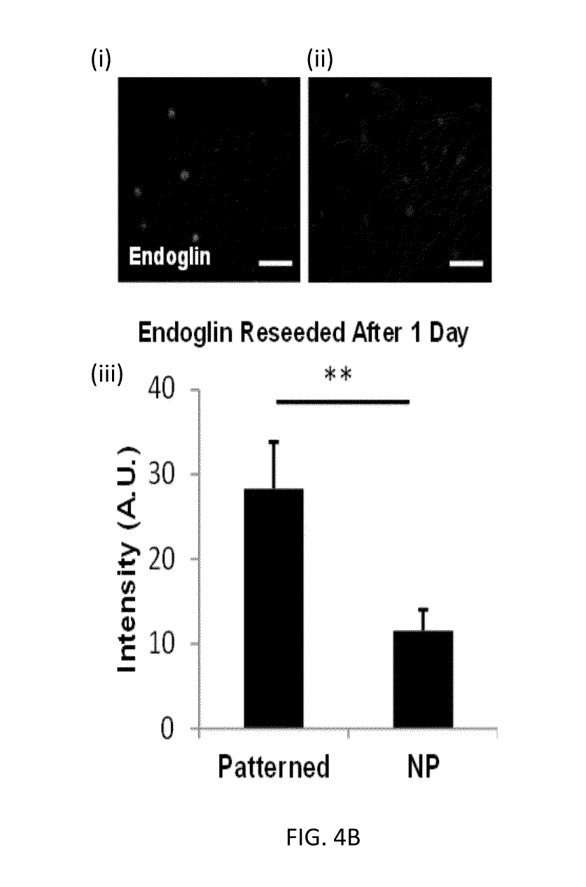

As shown in FIG. 4, mesenchymal stem cells show higher levels of multipotency markers (Stro-1 (FIG. 4A) and Endoglin (FIG. 4B) when cultured on patterned substrates compared to non-patterned substrates (sub-panels (iii) for both FIGS. 4A, B).

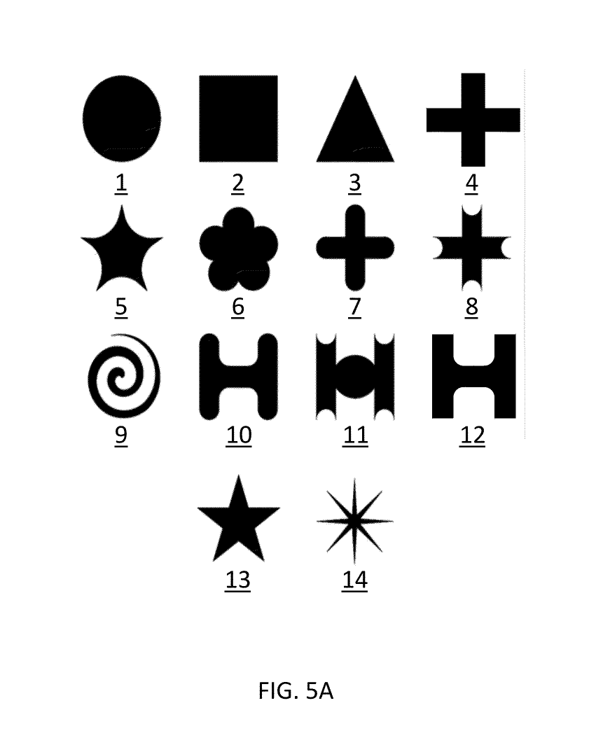

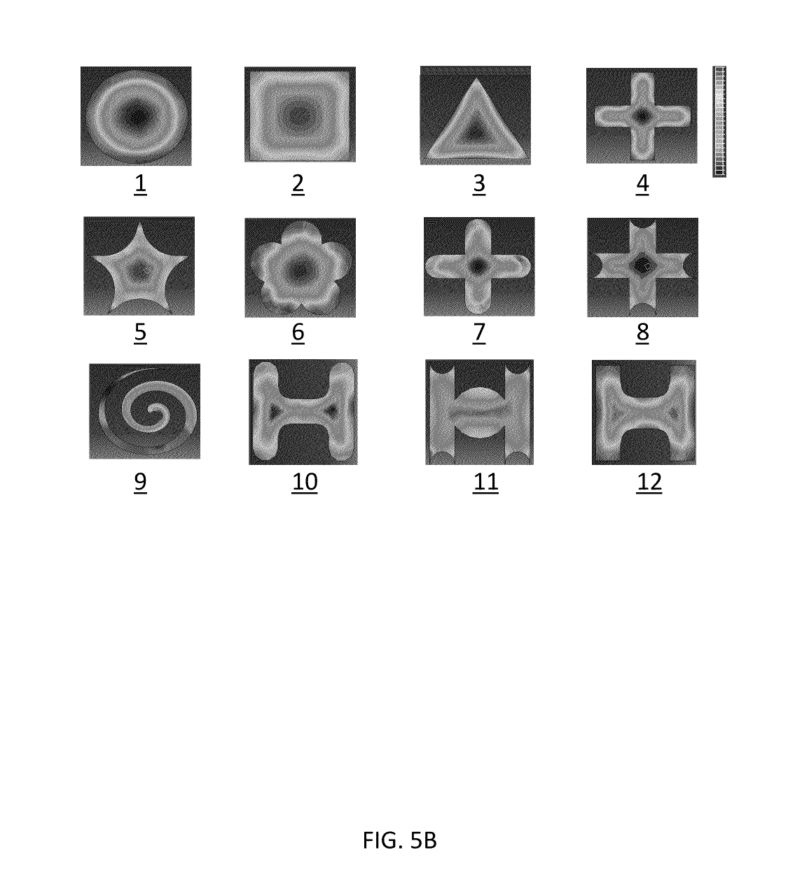

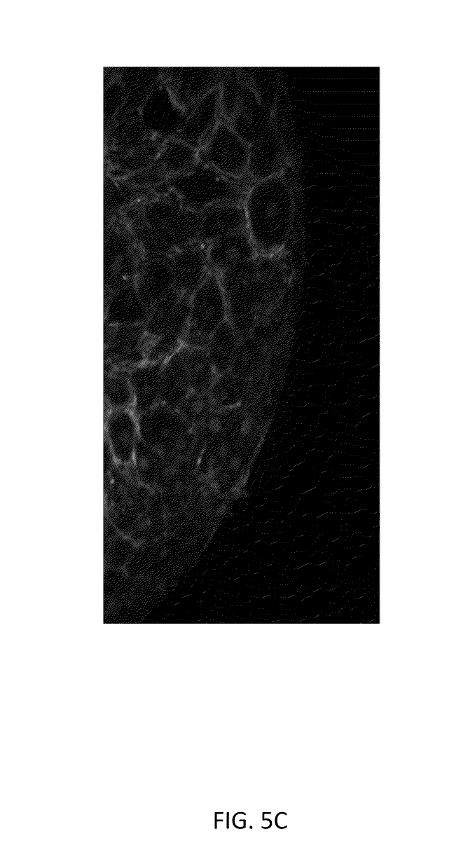

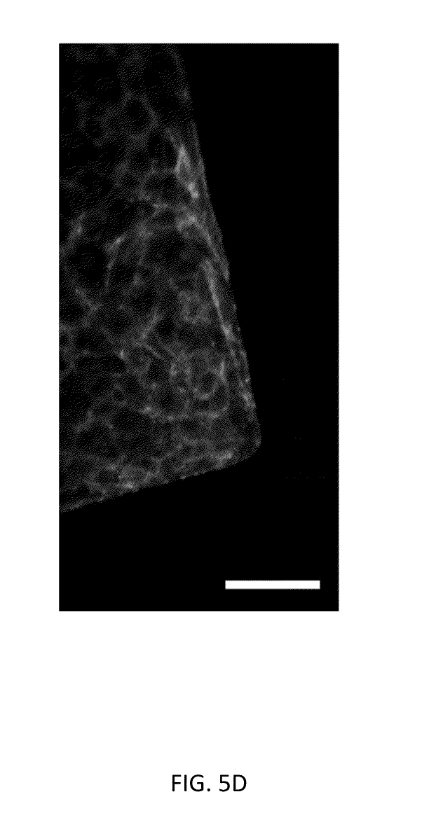

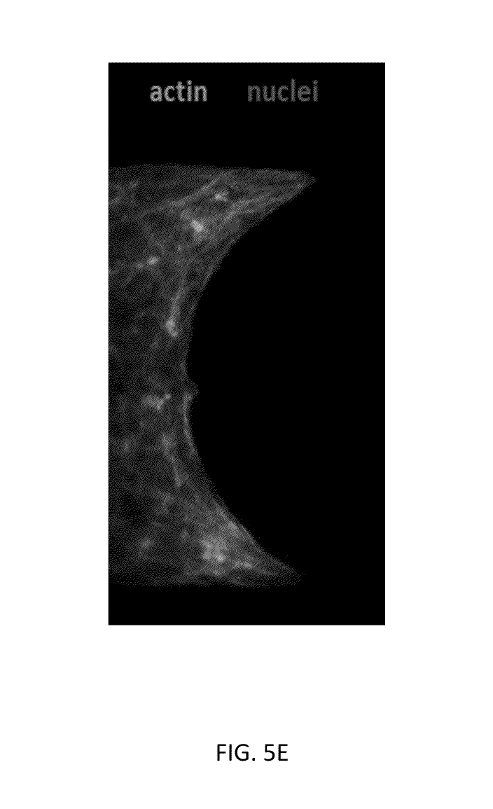

Referring to FIG. 5A, a variety of microengineered geometries were fabricated to evaluate the effect of specific micropatterned substrate geometries upon the effects of curvature and pinch points in guiding proliferation, chromatin architecture and METs. As used herein, “micropatterned object” refers to an exemplary geometry or shape fabricated on the substrate. Exemplary modeling of cellular tensions and/or compressions on a subset of these micropatterned objects using ABAQUS software is illustrated in FIG. 5B. The adhesion of mouse embryonic fibroblasts (MEFs) that contain a GFP gene in the Oct4 locus (Stemgent) were seeded on these microengineered surfaces. Stamps were fabricated with features of two different areas: 100,000 μm² and 1,000,000 μm² across a variety of shapes with different types of curvature that will present regions that impose variable degrees of mechanical force to the patterned cells. Passage 3 Oct4-GFP MEFs were thawed from cryopreservation and seeded at high density (approx. 100,000 cells/cm) onto the micropatterned substrates in growth media (high glucose DMEM containing L-glutamine, 10% ESC approved fetal bovine serum, and 1% penicillin-streptomycin). After four hours the remaining cells were aspirated and the surfaces were incubated at 37° C. and 5% CO₂. MEFs adhered uniformly across the patterns and remained geometrically confined for up to 2 weeks before they eroded the surface chemistry and proliferated across the surface (FIGS. 5C-E).

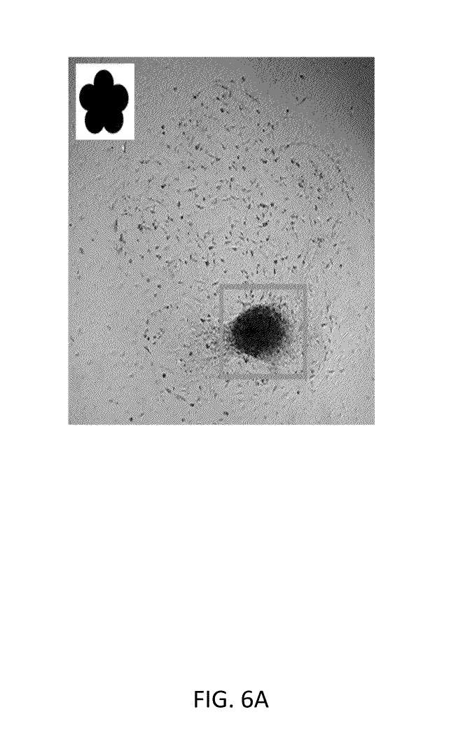

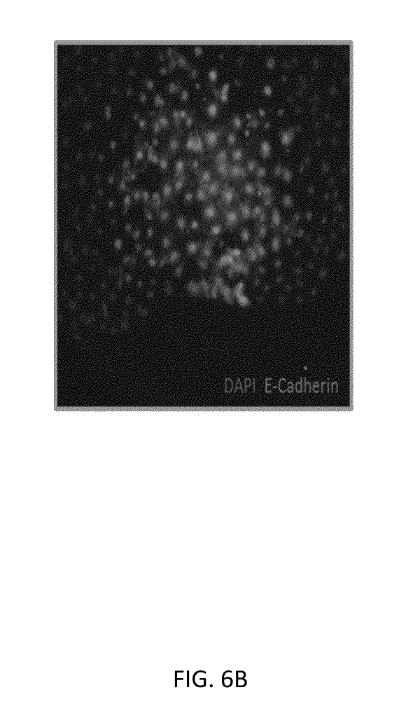

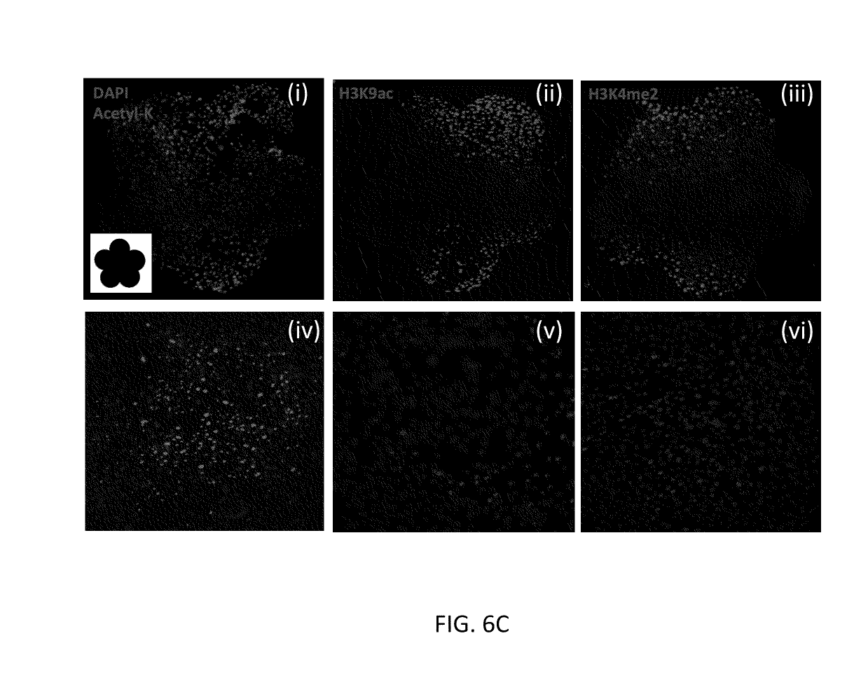

MEFs were cultured in growth media on geometrically patterned substrates for 1 week to determine the manner whereby geometric cues influence cellular packing, proliferation and the expression of markers associated with a MET. The cells were then fixed with 4% paraformaldehyde, permeablized with 0.1% Triton X-100 and blocked with 1% bovine serum albumin. Primary rabbit antibodies against mouse E-cadherin as a prototypical epithelial marker were applied to the surfaces overnight at 4 C followed by Alexa488-anti-rabbit, Texas Red X-phalloidin and DAPI for 1 hour at mom temperature. The unpatterned surfaces or regions of convexity showed background levels of E-cadherin. Interestingly, at these regions of concavity, a large increase in proliferation that leads to a budding of cells into a 3-D tightly packed spheroid are observed (FIG. 6A). Internal to regions of concavity, wherein one skilled in the art would expect actomyosin contractility to be low, expression of cytoplasmic E-cadherin is observed (FIG. 6B). Immunostaining demonstrates that the cells are viable. Thus, the disclosed method for substrate micropatterning enables one skilled in the art to engineer perimeter curvature to guide proliferation and MET towards enhancing somatic cell programming.

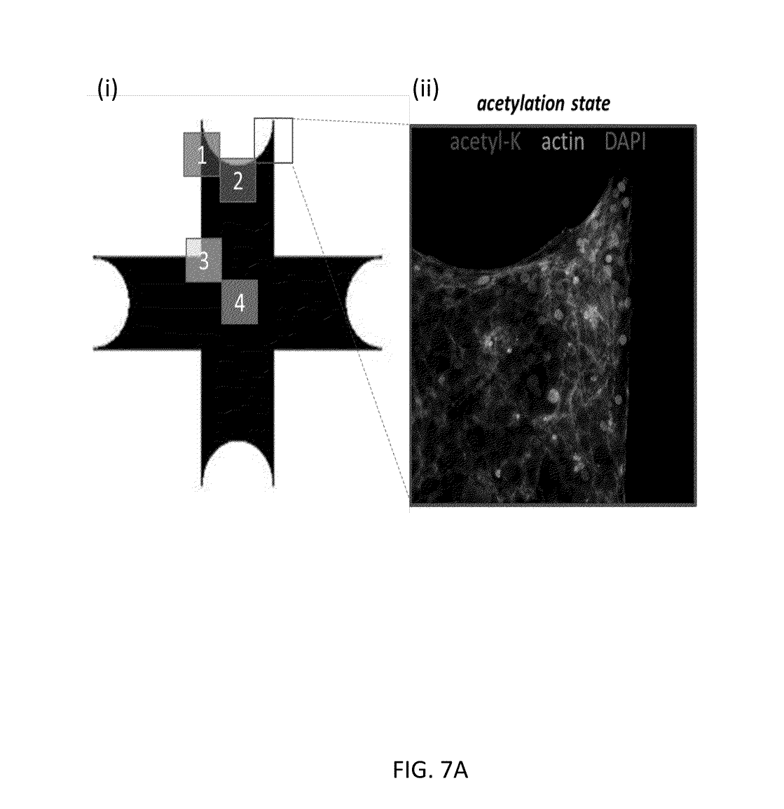

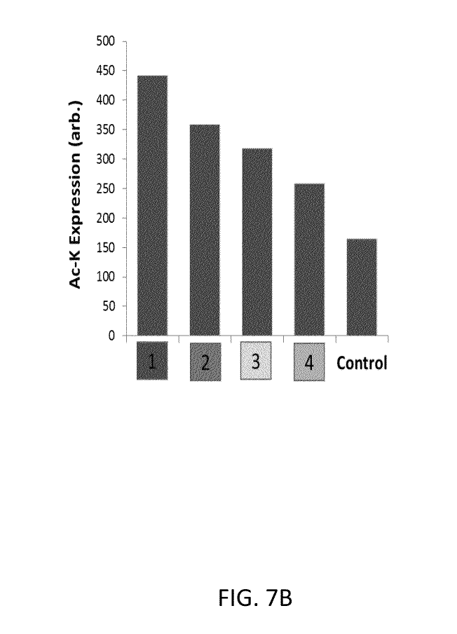

These geometric cues influence the acetylation state of nuclear histone proteins, as revealed in the following example. MEFs were cultured in the different patterns, fixed as described above and immunostained with rabbit anti-acetylated lysine followed by Alexa647-anti-rabbit, Alexa488-phalloidin and DAPI for 1 hour at room temperature. FIGS. 7A, B show pinch points at corners and regions of convexity at the pattern perimeter lead to enhancements in lysine acetylation. This result indicates that these geometric cues influence the balance of activities associated with histone acetylation state, which can lead to chromatin de-condensation and promote the removal of epigenetic marks that impede programming.

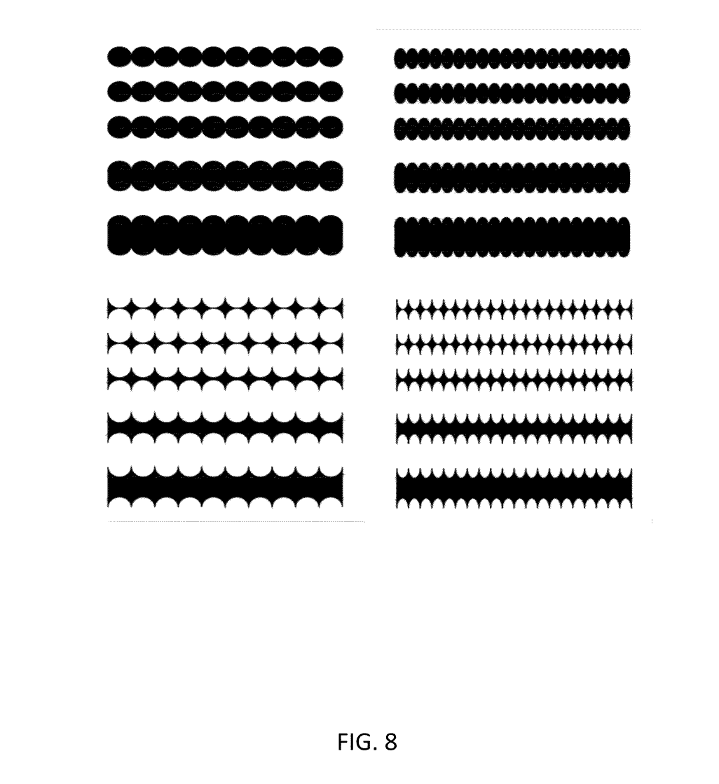

Geometric cues that influence cell morphological characteristics and packing across a surface can be discovered by fabricating surfaces presenting geometries with preferably four different total areas, such as the following: 10,000 μm², 50,000 μm², 100,000 μm² and 1,000,000 μm²in order to accommodate tens to tens of thousands of cells per shape. The choice of these pattern sizes is also motivated by the size of colonies observed during programming where initially, tens to hundreds of cells undergo a MET to initiate formation of a pre-iPSC colony. The assortment of exemplary geometric cues depicted in FIG. 5A is designed to present regions of curvature that are known to influence cell spreading, actomyosin contractility and compaction upon confluence. In addition to the fourteen exemplary shapes depicted in FIG. 5A, additional geometries fall within the scope of the disclosure that are limited only by the imagination of the skilled artisan in view of the present disclosure. Exemplary additional geometries includes shapes within shapes, such as circles within circles to provide a donut shape having an interior “hole,” an internal donut round area, and an external donut round area. In the foregoing examples, the shapes within shapes need not be the same basic geometric shapes. Thus, squares within circles; circles within squares; triangles within squares; as well as other shape with shape configurations are within the scope of the invention. Furthermore, geometries that show continuous patterns of different degrees of concavity and convexity with variable spacing between arcs can have utility in this regard (FIG. 8). MEFs can be transduced to express Oct4-GFP and seeded at high density as described above.

Polydimethylsiloxane stamps can be fabricated using soft-lithography for microcontact printing of alkanethiolates onto transparent gold substrates as described above. The intervening regions can be passivated with a non-fouling triethylene glycol diluent and matrix protein (fibronectin, vitronectin, collagen I and laminin) can be physisorbed to the hydrophobic regions for the capture of cells. MEFs that are transduced to express Oct4-GFP after de-differentiation to iPSCs are then seeded at high density on the patterned substrates in standard MEF growth media (DMEM with FBS) and with iPSC media (DMEM with LIF, β-mercaptoethanol and ESC qualified FBS). Cells are observed over time using phase contrast microscopy and fixed for immunofluorescence staining of E-cadherin (an exemplary MET marker), and acetylated lysine (an exemplary epigenetic marker).

Patterns that promote the highest expression of markers associated with MET and histone modifications can be selected for comparison with unpatterned controls through RNA and protein analysis by quantitative RT-PCR and western blot respectively. Table 1 shows a panel of exemplary markers that will be assessed using quantitative RT-PCR. Differential gene expression between the experimental and control conditions can be to guide the selection of antibodies for western analysis.

TABLE 1
Exemplary genes for RT-PCR analysis of patterned
and unpatterned cultures

| Mesenchymal | Epithelial | Pluripotency |
|---|---|---|
| Snail | E-cadherin | Oct4 |
| Vimentin | Entactin | Sox2 |
| Beta-catenin | Cytokeratin | Nanog |
| Fibronectin | Laminin-1 | Esrrb |
| N-cadherin | Epcam | Lin28 |

### Compositions and Fabrication Methods for Micro-Patterned Substrates Having Tunable Stiffness

The mechanical properties of the substrate can be tuned to enhance cell-cell adhesion and promote the epithelial phenotype in patterned mouse embryonic fibroblasts. Glycoproteins can be covalently patterned onto polyacrylamide gels of physiologically relevant stiffness (for example, E in the range from about 0.1 kPa to about 1 MPa) (FIG. 3D). Briefly, and with reference to FIG. 3D, glass slides were modified with APTES and gluteraldehyde followed by polymerization of different combinations of acrylamide and bis-acrylamide to target physiologically relevant stiffness ranges. The gels were immersed in hydrazine hydrate for 2 hours followed by aspiration and thorough rinsing. Sodium periodate was used to oxidize fibronectin, (25 μg/mL) for 45 minutes followed by application to PDMS stamps fabricated as described above for 10 minutes. Stamps were briefly dried under a stream of nitrogen then brought into contact with the semi-dried hydrogel surfaces for 2 minutes. The modified surfaces were transferred to sterile PBS in a 12-well plate and Oct4-GFP MEFs were seeded onto the gels at 50,000 cells/mL.

### Cell Programming on Micro-Patterned Substrates Having Tunable Stiffness

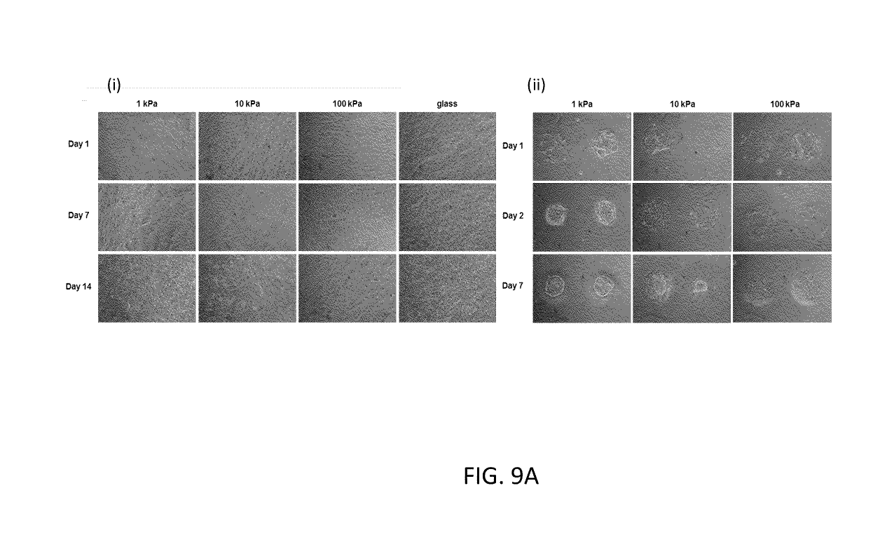

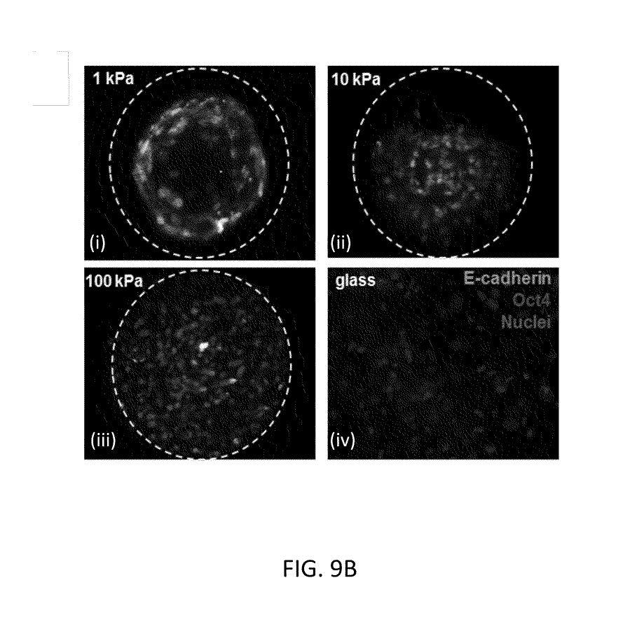

The morphological characteristics of cells cultured on both patterned and unpatterned hydrogel substrates were studied over time. After initial adhesion the cells adopted spindle shapes characteristic of MEFs across all three stiffness gels (FIG. 9A). However, after several days cells that were cultured in patterns on the softest gels began to develop morphological characteristics that are reminiscent of epithelial packing. These cells were immunostained for E-cadherin as described above. MEFs cultured on the 1 kPa gels after 2 weeks under growth conditions showed a colony-like morphology retracted from the patterned perimeter with enhanced E-cadherin expression (FIG. 9B). This result indicates that tuning substrate mechanics can be used to promote METs in patterned populations of MEFs.

Geometric cues and matrix mechanics increase programming efficiency by directing chromatin de-condensation and METs at the early initiation phase. Circular geometries (150,000 μm²) showing epithelial-like packing after 1 week demonstrate this principle. Patterned substrates were fabricated by microcontact printing of octadecanethiol onto thin layers of transparent gold followed by passivation in triethylene glycol undecanethiol. Fibronectin was physisorbed to the hydrophobic regions for the capture of cells. Passage 3 MEFs were seeded at high density on the patterned substrates in standard MEF growth media (DMEM with FBS).

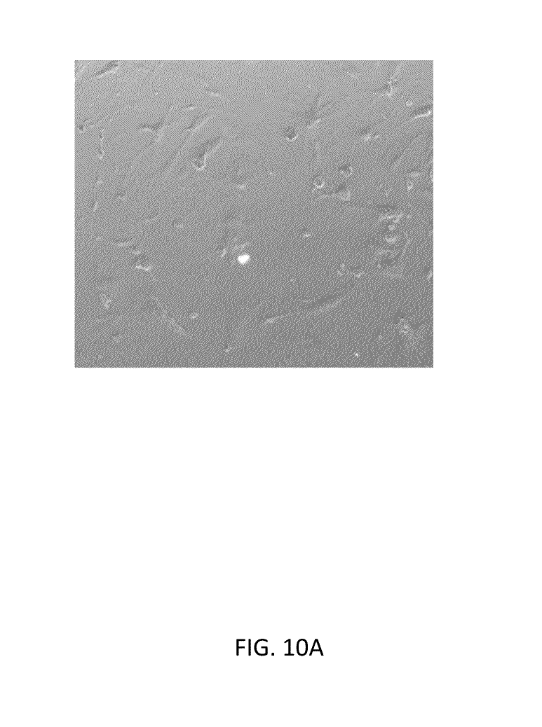

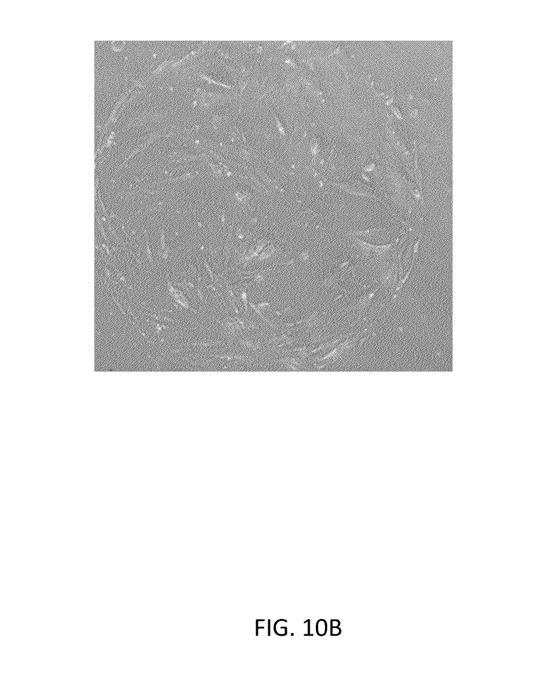

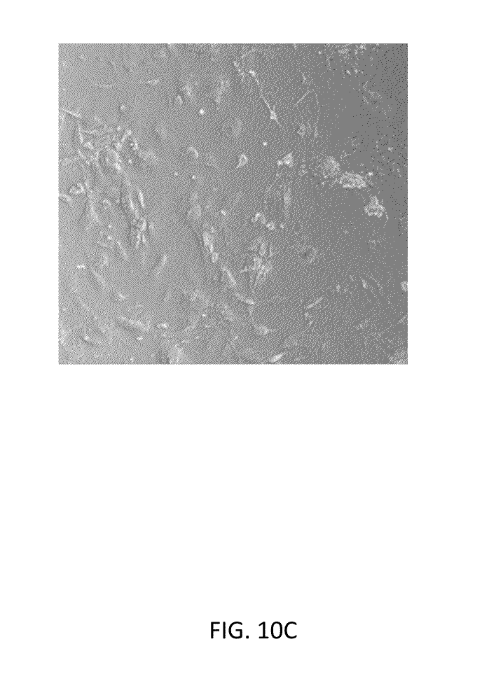

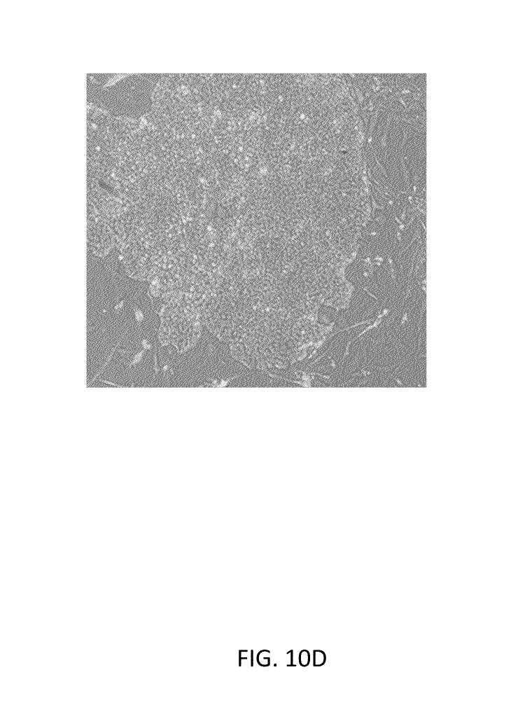

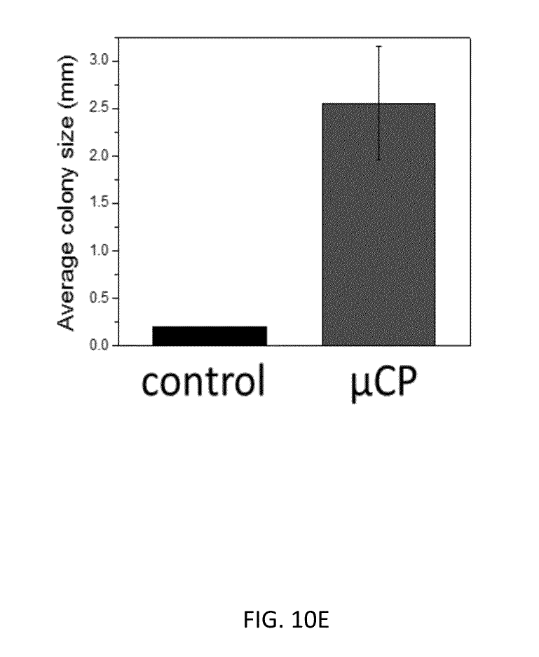

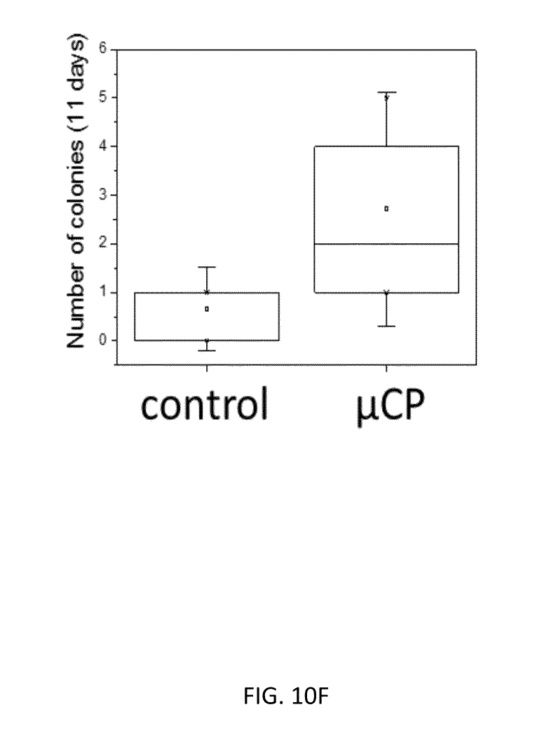

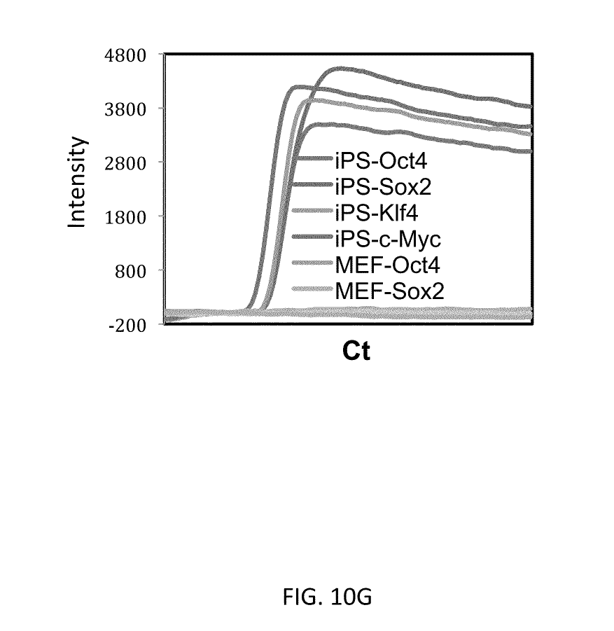

Plasmids containing the four doxycycline inducible Yamanaka factors Oct4, Sox2, cMyc and Klf4, the tetracycline controllable transactivator gene M2rtTa, and lentivirus packaging and encapsulation genes were bought from Addgene contained in a bacterial vector with ampicillin resistance. Bacteria were then grown in lysogeny broth containing ampicillin and plasmids purified with a Qiagen Plasmid Maxi kit and confirmed with PCR. Lentiviral packaging plasmid psPAX2, envelope plasmid pMD2.G, and a plasmid containing the gene of interest were then transfected into HEK 293 packaging cells with Fugene. Lentivirus were harvested at day four and confirmed with Lenti-X qRT-PCR Titration Kit from Clontech. The quantity of each virus was normalized after titer and added to Oct4-GFP MEFs cultured on gelatin-coated plastic and our patterned surfaces. The media was exchanged for ESC media containing LIF and doxycycline and changed every two days. The MEFs degraded the surface chemistry and escaped the patterned regions after 1 week; however, after 11 days in culture, the appearance of colonies with the characteristic morphology of iPS cells in both patterned and unpatterned control conditions were noted. Strikingly, cultures where cells were previously micropatterned led to more and bigger colonies suggesting that these geometric cues may influence the programming process (see FIGS. 10A-G).

Transformation of programmed cells can also be accomplished by including soluble additives to culture media used for feeding the programmed cells. Exemplary transformation protocols are well known in the art. See, for example, recombinant proteins (Zhou, H. et al., “Generation of induced pluripotent stem cells using recombinant proteins,” Cell Stem Cell. 4:381-4 (2009)), mRNA (Warren, L. et al., “Highly Efficient Reprogramming to Pluripotency and Directed Differentiation of Human Cells with Synthetic Modified mRNA,” Cell Stem Cell. 7:618-30 (2010)), microRNAs (Li, Z. et al., “Small RNA-mediated regulation of iPS cell generation,” EMKO J. 30:823-34 (2011); Anokye-Danso, F. et al., “Highly Efficient miRNA-Mediated Reprogramming of Mouse and Human Somatic Cells to Pluripotency,” Cell Stem Cell. 8:376-88 (2011)), and small molecules that target chromatin modifying enzymes and other signaling pathways (Yuan, X. et al., “Small molecules in cellular reprogramming and differentiation,” Prog. Drug Res. 67:253-66 (2011); Feng, B. et al., “Molecules that promote or enhance reprogramming of somatic cells to induced pluripotent stem cells,” Cell Stem Cell. 4:301-12 (2009); Hou, P. et al., “Pluripotent Stem Cells Induced from Mouse Somatic Cells by Small-Molecule Compounds. Science,” 341:651-4 (2013)).

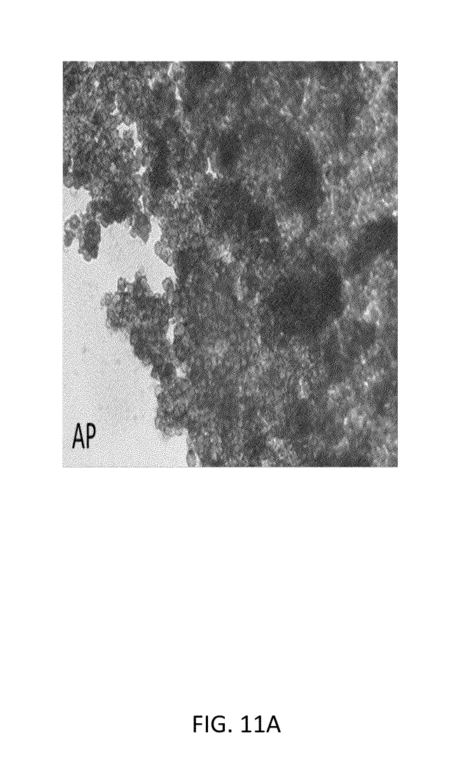

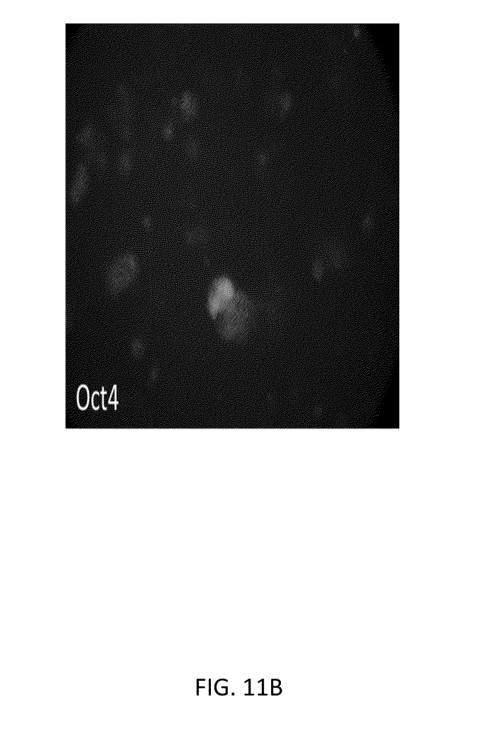

Analyses of proliferation rates and pluripotency marker staining patterns are preferred methods to characterize the iPSCs generated on these surfaces. These cells showed enhanced division as demonstrated through doubling times of approximately 12 hours, consistent with iPSCs and mESC biology. Furthermore, these cells expressed endogenous Oct4 and stained positive for alkaline phosphatase (FIG. 11A, B). However, Oct4 expression was not uniform in these cells that suggest pre-iPS populations that require stabilization through the addition of 2i media or expansion on feeder cells.

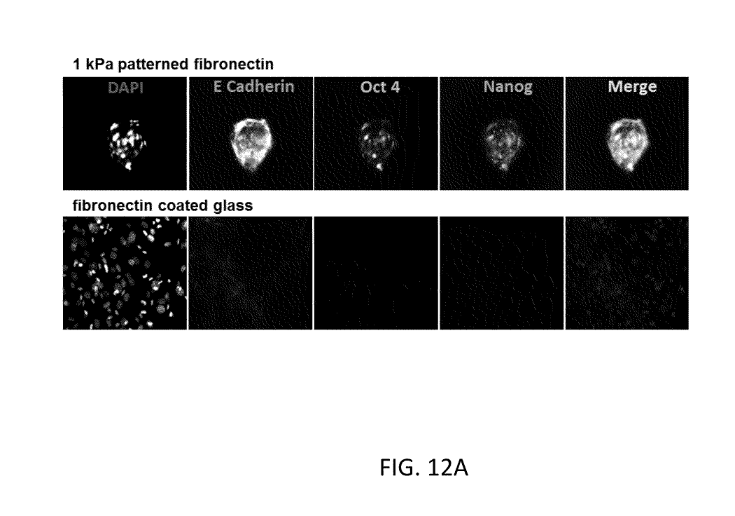

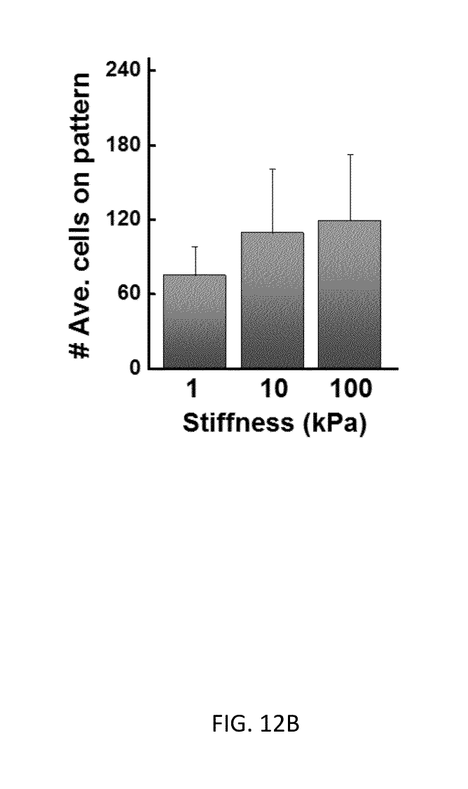

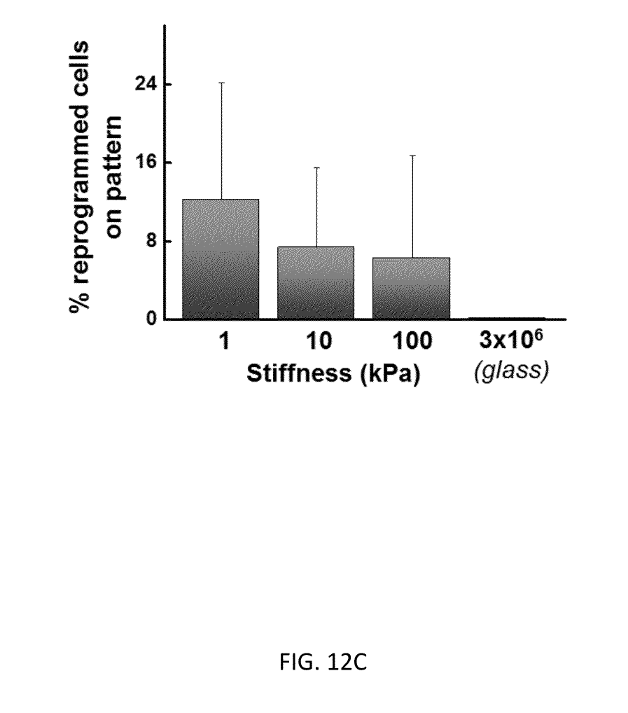

In conjunction with the patterning on gold surfaces, the same circular geometry of 150,000 μm² on polyacrylamide hydrogels of stiffness spanning the physiological range of 1, 10 and 100 kPa was evaluated with rigid glass (10⁶ kPa) as control. The gels were first treated with hydrazine hydrate and oxidized fibronectin was microcontact printed onto the surface. Oct4-GFP MEFs were seeded onto the gels at ˜50,000 cells/mL. After 1 week in culture the patterned cells on 1 kPa gels displayed morphological characteristics consistent with colony formation-3-D spheroids of cells demonstrating compact nuclei compared to cells cultured on unpatterned glass. The nuclear characteristics of these cells are consistent with previous studies demonstrating significant differences between chromatin compaction between MEFs and ESCs (FIG. 12A). The cells were fixed on all surfaces with 4% PFA, permeabilized with Triton X-100, blocked with BSA and performed four color immunofluorescence analysis with markers for E-cadherin, Oct4, Nanog and DAPI for nuclei. FIG. 12B shows immunofluorescence images for patterned MEFs on the 1 kPa gels compared to fibronectin coated glass. Cells that stained positive for all three markers were counted as positive for programming; FIG. 12C shows how all three stiffness gels lead to programmed cells with the highest on 1 kPa (˜13%)>10 kPa (˜7%)>100 kPa (˜6%)>>glass (<1%). These results provide compelling evidence that patterned hydrogel matrices enhance programming with lentivirus transduction compared to a glass control.

### Kits

In another aspect, kits are provided for preparing the micropatterned substrates and methods for their use in cell programming as described herein. Kits can come in two forms. A first kit is amenable for making the micropatterned substrates. Such kits can include suitable substrates already chemically modified or with a suitable matrix having a tuned stiffness, stamps, chemical imprinting medium, inert medium for passivation, cell adhesion agents and instructions for making the micropatterned substrates. A second kit is amenable for using premade micropatterned substrates for cell programming. Such kits can include, premade micropatterned substrates, control unpatterned substrates, optionally, adhesion agents in separate packaging if the adhesion agent is not already present on the premade substrates, immunohistochemical stains and other reagents for detecting epigenetic marker, MET markers and the like, optionally, iPSC gene transduction reagents, as well as instructions.

### Examples

### Example 1

### Patterning Stem Cells Promotes Multipotency

We developed methods to explore how restricting cell spreading through patterning would influence the programming of heterogeneous mesenchymal stem cells (MSCs) to multipotent MSCs. Human MSCs and differentiation media were purchased from Lonza (Basel, CH). These cells were derived from bone marrow isolated from the iliac crest of human volunteers. MSCs were tested for purity by Lonza, and were positive for CD105, CD166, CD29, and CD44, negative for CD14, CD34, and CD45 by flow cytometry, and had ability to differentiate into osteogenic, chondrogenic, adipogenic lineages. Surfaces were fabricated by electron beam evaporation of 5 nm of Ti followed by 20 nm of Au onto cleaned glass coverslips. To create patterned surfaces, PDMS (Polysciences, Inc.) stamps were fabricated by polymerization upon a patterned master of photoresist (SU-8, MicroChem) created using UV photolithography through a laser printed mask. Stamps featuring circular patterns of 1000 μm² were used. Stamps were inked with 10 mM octadecanethiol in ethanol, dried under air, and applied to the surface. Surfaces were then incubated overnight with 3 mM tri(ethylene glycol) undecanethiol in ethanol to prevent protein adsorption and cell adhesion to non-patterned regions. Next, 50 μg/mL fibronectin was applied to surface for 1 h at room temperature. For non-patterned surfaces, 50 μg/mL fibronectin was applied to gold coverslips for 1 h. Surfaces were rinsed with PBS and stored in PBS until use. MSCs were thawed from cryopreservation (10% DMSO) and cultured in Dulbecco's Modified Eagle's Medium (DMEM) low glucose (1 g/mL) media supplemented with 10% fetal bovine serum (MSC approved FBS; Invitrogen), 1% penicillin/streptomycin, media changed every 3-4 days and passaged at ˜80% confluency using Trypsin:EDTA (Gibco). Passage 4-8 MSCs were seeded on patterned and non-patterned surfaces at a cell density 5000 cells/cm². After one week of culture on patterned or non-patterned substrates, the surfaces were gently transferred to new 12-well plates. Cells were trypsinized and reseeded into 24-well plates with basal media (DMEM supplemented with 10% FBS, 1% penicillin/streptomycin).

Cell proliferation was assessed via BrdU staining. 20 fields of view were taken for every sample condition. Quantification was performed using ImageJ. After 1 h postseeding, non-adherent cells were aspirated and BrdU labeling reagent (Invitrogen) was added to DMEM supplemented media at a concentration of 1:100 (v/v), and incubated 24 h. After washing with PBS, cultures were fixed in 70% ethanol for 30 min followed by PBS rinsing. Cultures were then denatured with 2M HCl for 30 min. followed by PBS rinsing. Cultures were permeabilized with 0.1% Triton X-100 in PBS for 30 min. and blocked with 1% BSA in PBS for 15 min. Cultures were then incubated with mouse anti-BrdU primary antibody (1:200 dilution, 3 hours at mom temperature) followed by Alexa Fluor 647-conjugated anti-mouse IgG antibody (1:200 dilution, 1 hour at mom temperature). Cell nuclei were stained with DAPI (1:5000 dilution). Images of the DAPI and 647 channels were overlayed and percent incorporation of BrdU was counted manually.

For assessing multipotency we performed immunofluorescence staining for mesenchymal markers Endoglin/CD105 and Stro-1 (FIG. 4). Cells were fixed with 4% paraformaldehyde for 20 minutes, permeabilized with 0.1% Triton X-100 in PBS for 30 minutes, and blocked with 1% BSA for 15 minutes. Primary antibody labeling was performed in 5% goat serum containing 1% BSA in PBS overnight at 4° C. with rabbit anti-Endoglin (Sigma, 1:200 dilution) and mouse anti-Stro-1 (R&D Systems, 1:200 dilution). Secondary antibody labeling was performed similarly with Tetramethylrhodamine-conjugated anti-rabbit IgG antibody and Alexa Fluor 647-conjugated anti-mouse IgG antibody (1:200 dilution) along with Alexa Fluor 488-phalloidin (1:200 dilution) and DAPI (1:5000 dilution) for 1 hour at mom temperature. Immunofluorescent images were analyzed using ImageJ. Regions of interest were selected by outlining actin filaments for non-patterned surfaces, or by manually selecting patterned cells. For the regions of interest, images were thresholded to select positively stained areas and integrated density (representing mean gray scale times feature area) was calculated. DAPI staining was used to count nuclei. Integrated density was totaled for each condition and normalized to cell number to give intensity per cell (arbitrary units). At least two independent experiments each with duplicate samples were performed to verify results. Each condition analyzed represent 100-3000 cells. Increased expression of CD105 and Stro-1 indicated that the patterning procedure converted the heterogeneous cells to a homogenous multipotent stem cell population. Imaging was performed using a GE InCell Analyzer 2000 at 20× magnification.

### Example 2

### Patterning Mouse Embryonic Fibroblasts Enhanced Programming to Induced Pluripotent Stem Cells

We designed large geometries with variable curvature at the perimeter that promote variable regions of tension and compression on large populations of cells. A finite-element model of contractile cell monolayers was built based on ABAQUS FEA software (Nelson, C. M. et al. “Emergent patterns of growth controlled by multicellular form and mechanics,” Proc. Natl. Acad. Sci. U.S.A. 102, 11594-9 (2005)). A model with the designed geometry was constructed consisting of 2 layers: an active 20 um thickness top layer and a passive 5 um bottom layer fixed at the bottom surface. Contractility was introduced to the active layer by applying a 5K temperature drop to promote isotropic thermal strain. Testing multiple mesh sizes and layer properties was performed for the convergence of results (FIG. 5B).

To pattern the cells we generated silicon masters by photolithography using a laser printed mask onto 20-30 μm thick layer of photoresist (SU-8, Microchem) and PDMS stamps were fabricated upon this template. Guided by our finite element analysis we chose to initially pattern circular geometries that display a uniform distribution of perimeter tension and interior compression; stamps were prepared with feature sizes ranging from 50,000-150,000 μm² to accommodate hundreds to thousands of cells. Surfaces were prepared by electron beam evaporation of titanium (5 nm) followed by gold (20 nm). Next we inked our PDMS stamps with 1 mg/ml octadecanethiol in ethanol, followed by drying under a stream of compressed air, and brining the stamp into contact with the gold surface to define the pattern. The patterned surfaces were incubated in 1 mM solution of triethylene glycol undecanethiol (Sigma) at room temperature overnight. Surfaces were rinsed with ethanol, dried under compressed air and set face-down on 25 μg/ml fibronectin in phosphate buffered saline (PBS) for 1 hour. Surfaces were transferred directly to 12-well tissue culture plates under PBS and stored until use.

Oct4-GFP MEFs (passage 3, Stemgent) were thawed from cryopreservation and seeded at 250,000 cells/cm² onto the patterned substrates and cultured for 7 days in high glucose Dulbecco's modified eagles medium (DMEM). Media was aspirated and replaced with 4% paraformaldehyde for 20 minutes, followed by incubation in 1% BSA (v/v) for 1 hour, and 0.1% Triton X-100 for 1 hour. Cells were immunostained for mouse E-cadherin (1:500) (abcam) and rabbit acetylated lysine (1:500) (cell signaling technologies) in 1% BSA overnight at 4C. Secondary antibody labeling was performed using Alexa488-anti-rabbit, Alexa647-anti-mouse, Texas Red X-phalloidin and DAPI for 1 hour at mom temperature. Immunofluorescence microscopy was conducted using a Zeiss Axiovert 200 M inverted research-grade microscope (Carl Zeiss, Inc.) or a LSM 700 (Carl Zeiss, Inc.) which is a four laser point scanning confocal with a single pinhole. FIG. 6 shows a flower-shape geometry (FIG. 6A) and a cross-shape geometry (FIG. 6B) immunostained for E-cadherin and acetylated-lysine. Interstitial points between the petals of the flower show a low degree of stress through the model and on these spots after 7 days, MEFs show increased E-cadherin expression. Points at the perimeter of patterns like in the cross example lead to higher degrees of acetylation in the nucleus. We also immunostained for histone marks involved in programming, H3K9ac and H3K4me2, and found that cells on regions of perimeter curvature showed higher levels of these epigenetic marks (FIG. 6C).

Plasmids containing the four doxycycline inducible Yamanaka factors Oct4, Sox2, cMyc and Klf4, the tetracycline controllable transactivator gene M2rtTa, and lentivirus packaging and encapsulation genes were bought from Addgene contained in a bacterial vector with ampicillin resistance. Bacteria were then grown in lysogeny broth containing ampicillin and plasmids purified with a Qiagen Plasmid Maxi kit and confirmed with PCR. Lentiviral packaging plasmid psPAX2, envelope plasmid pMD2.G, and a plasmid containing the gene of interest were then transfected into HEK 293 packaging cells with Fugene. Lentivirus were harvested at day four and confirmed with Lenti-X qRT-PCR Titration Kit from Clontech. The quantity of each virus was normalized after titer and added to Oct4-GFP MEFs cultured on gelatin coated plastic and our patterned surfaces. The media was exchanged for ESC media containing LIF and doxycycline and changed every two days. The MEFs degraded the surface chemistry and escaped the patterned regions after 1 week; however, after 11 days in culture we noted the appearance of colonies with the characteristic morphology of iPS cells in both patterned and unpatterned control conditions. Cultures where cells were previously micropatterned led to more and bigger colonies suggesting that these geometric cues may influence the programming process (FIG. 10). To characterize the iPS cells generated on our surfaces we characterized proliferation rates and stained for pluripotency markers. These cells showed enhanced division as demonstrated through doubling times of approximately 12 hours, consistent with iPSCs and mESC biology. Furthermore, these cells expressed endogenous Oct4 and stained positive for alkaline phosphatase after incubation in BCIP/NBT solution (Amresco (Solon, Ohio (US)) for 20 minutes (FIG. 11). We also performed quantitative RT-PCR of the starting population and the iPSCs generated on our surfaces. Adherent cells were lysed directly in TRIZOL reagent (Invitrogen) and total RNA was isolated by chloroform extraction and ethanol precipitation. Total RNA in DEPC water was amplified using TargetAmp™ 1-Round aRNA Amplification Kit 103 (Epicentre) according to vendor protocols. Total RNA was reverse transcribed using Superscript III® First Strand Synthesis System for RT-PCR (Invitrogen). RT-PCR was performed linearly by cycle number for each primer set using SYBR® Green Real-Time PCR Master Mix (Invitrogen) on an Eppendorf Realplex 4S Real-time PCR system. FIG. 10G shows Ct traces for Oct4, Sox2, Klf4, and c-Myc demonstrating robust expression at the mRNA level of all four factors.

### Example 3

### Constraining Mouse Embryonic Fibroblasts in Circular Shapes on Hydrogels of Physiological Stiffness Enhance Mesenchymal to Epithelial Transitions and Expression of Pluripotency Markers

One-half percent 3-aminopropyltriethoxysilane (APTs) and 0.5% glutaraldehyde (Gluta) were employed to modify the surface chemistry of a coverslip (18 mm). The procedure of fabricating hydrogels was followed by the previous report (Tse, J. R. & Engler, A. J. “Preparation of hydrogel substrates with tunable mechanical properties,” Curr. Protoc. Cell Biol. 47, 10.16.1-10.16.16 (2010)). Briefly, the mixture of 5, 10, or 10% of Acrylamide (Amresco Inc.) and 0.03, 0.1 or 0.9% of Bis-acrylamide (Amresco Inc.) were used to prepare 1, 10, or 100 kPa polyacrylamide hydrogels, respectively. As initiators 0.1% of ammonium persulfate and tetramethylethylenediamine were applied for the polymerization. The mixture (20 μl) was placed on the hydrophobic treated glass slides and the surface treated coverslip was put on it with the treated side down. The polymerized gels on coverslip were gently detached after suitable polymerization time depending on the stiffness of gels (˜20-25 min) and the stiffness were confirmed by measuring elastic moduli of gels based on atomic force microscopy (AFM) contact force measurements on atomic force microscope (Asylum Research) (Lee, J., Abdeen, A. a., Huang, T. H. & Kilian, K A “Controlling cell geometry on substrates of variable stiffness can tune the degree of osteogenesis in human mesenchymal stem cells,” J. Mech. Behav. Biomed. Mater. (2014) doi:10.1016/j.jmbbm.2014.01.009). The surface chemistry of gels were changed from amide groups to reactive hydrazide groups by using hydrazine hydrate 55% (˜2 hours, Fisher Scientific) (Damljanović, V., Lagerholm, B. C. & Jacobson, K. “Bulk and micropatterned conjugation of extracellular matrix proteins to characterized polyacrylamide substrates for cell mechanotransduction assays,” Biotechniques 39, 847-851 (2005)). 5% glacial acetic acid (1 hour, Fluka/Sigma) and distilled water (1 hour) were employed to wash the gel surface. Sodium periodate (˜3.5 mg/ml, Sigma-Aldrich) employed to generate free aldehydes was applied to 25 μg/mL of mixture of fibronectin, laminin, and collagen I (1:2:2 ratio) in PBS at least 45 min (Lee, J., Abdeen, A. A., Zhang, D. & Kilian, K. A. “Directing stem cell fate on hydrogel substrates by controlling cell geometry, matrix mechanics and adhesion ligand composition,” Biomaterials 34, 8140-8 (2013)). The protein solution was pipetted onto patterned or non-patterned stamps for 30 min. These stamps were produced by conventional polymerization methods of PDMS on patterns with photoresist (SU-8, Micro-Chem) created using UV photolithography through a laser printed mask or unpatterned (flat) surfaces. Cured stamps were gently detached from the molds. The protein solution was dried with air and then the protein residues on stamps were transferred to the gel surface; free aldehydes in proteins were chemically conjugated with reactive hydrazide groups on the gels. APTs and Gluta treated coverslips were added on the solution (on parafilm) for around 1 hour with the treated surface down and used as a glass control (FIG. 8).

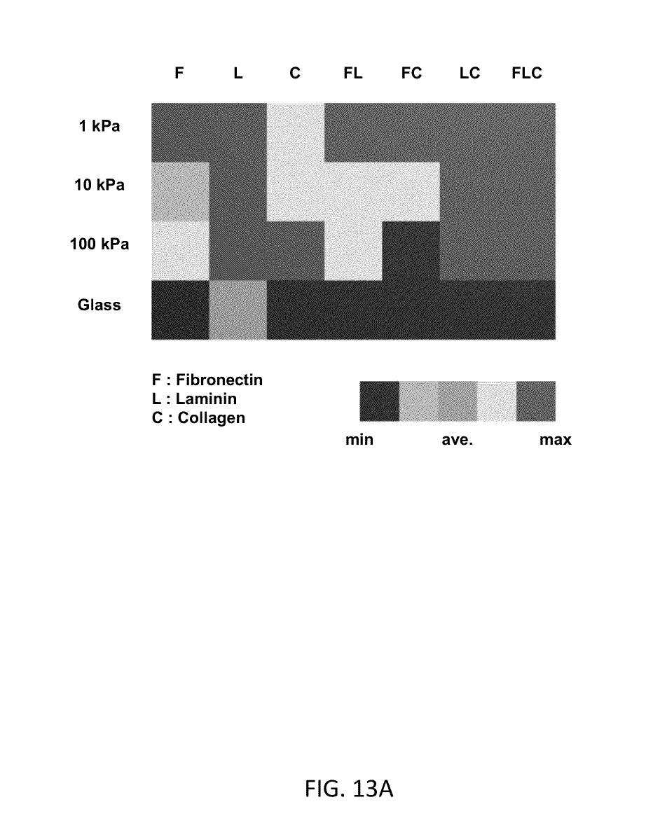

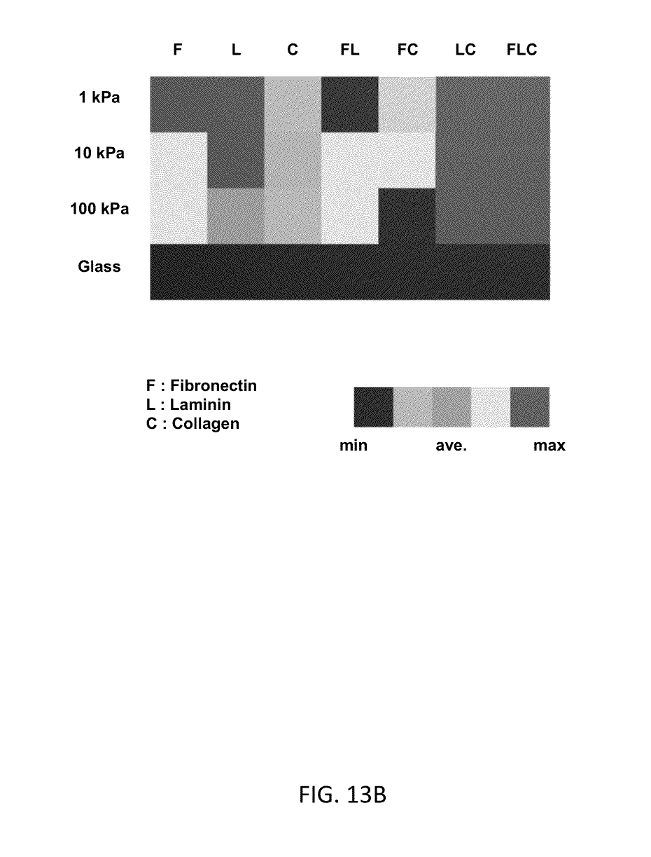

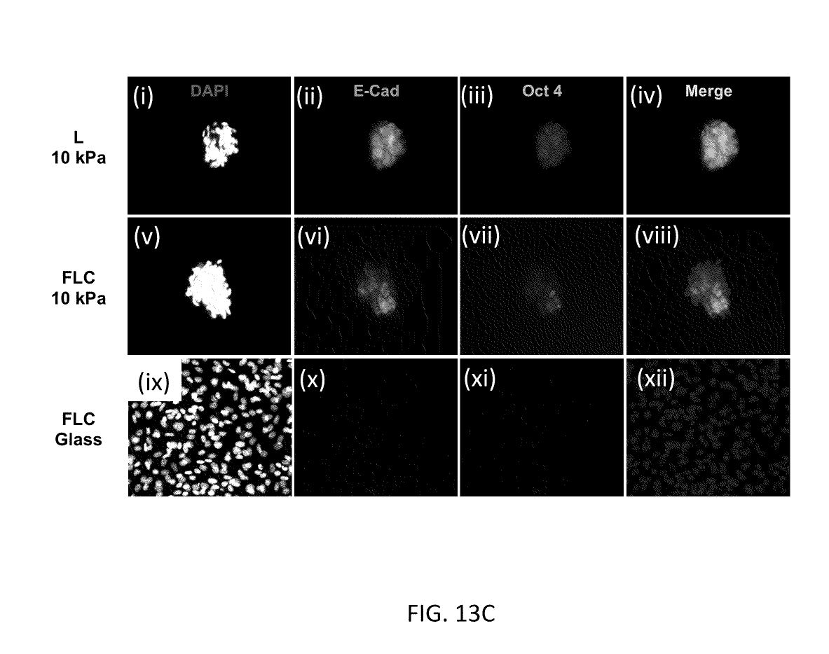

Mouse embryonic fibroblasts (primary MEFs isolated from C57BL/6 mice) were cultured in Dulbecco's Modified Eagle's Medium (DMEM, Corning) high glucose (4.5 g/L) media mixed with 15% fetal bovine serum (HI FBS, Gibco), and 1% penicillin/streptomycin (p/s, Gibco). Media was changed every 3 days and cells were passaged at nearly 80% confluency using 0.25% Trypsin:EDTA (Gibco). MEFs at passage 2 or 3 were seeded on patterned (150,000 μm²) and non-patterned surfaces at a cell density at least 10,000 cells/cm² and then the media was changed 1 hour after seeding, so that non-adherent cells were aspirated. After incubation for a specified time point, surfaces were fixed with 4% formaldehyde (Ted Pella, Inc.) for 20 min and permeabilized in 0.1% Triton X-100 in PBS for 30 min and then blocked with 1% bovine serum albumin (BSA) for 15 min. Primary antibodies were labeled in 1% BSA in PBS for 2 hours at mom temperature (20° C.) with rabbit anti-E-cadherin (Cell signaling_{—}3195P, 1:500 dilution) or N-cadherin (Cell signaling_{—}4061P, 1:500 dilution), mouse anti-SSEA (Abcam_ab16285, 1:200 dilution), acetylated lysine (Cell signaling_{—}9441S, 1:200 dilution), E-cadherin (Abcam_ab1416, 1:500 dilution), or Nanog (Sigma_N4413, 1:200 dilution), and goat anti Oct4 (Abcam_ab27985, 1:500 dilution). Second antibody labeling was accomplished using Alexa Fluor 555-conjugated anti-mouse IgG antibody (Life technologies, 1:200 dilution) or donkey anti-Goat IgG (Cy3) antibody (Abcam_ab6949) and Alexa Fluor 647-conjugated anti-mouse IgG antibody (1:200 dilution) along with Alexa Fluor 488-phalloidin (Life technologies, 1:200 dilution) and DAPI (1:5000 dilution) for 20 min in the humid chamber (37° C. with 5% CO₂). Immunofluorescence microscopy was conducted using a Zeiss Axiovert 200 M inverted research-grade microscope (Carl Zeiss, Inc.) or a LSM 700 (Carl Zeiss, Inc.) which is a four laser point scanning confocal with a single pinhole. FIG. 9 shows phase contrast images of MEFs that were cultured for different time points, on substrates of variable stiffness, with and without patterning constraints. FIG. 9B shows how patterning MEFs on soft hydrogels (˜1 kPa) leads to enhanced E-cadherin expression, and FIG. 12 shows how cells cultured on hydrogels for two weeks display the expression of pluripotent transcription factors. FIGS. 13A, B and C show how the expression of E-cadherin and Oct4 is influenced by the matrix protein conjugated to the surface where inclusion of laminin appears to be important for regulating the expression of these markers. At least, three repeats were done for each experimental group; the number of colony-like shapes was counted by manually at least 100 patterns per each condition and at least 30 patterns per each condition were measured for the quantitative analysis of relative marker intensity unless indicated otherwise. The backgrounds for each condition were subtracted to obtain intensity value for each cell from cytoplasmic or nuclei staining intensity (depending on each marker). Statistical significance was determined via Student's t test, where p<0.05. The data is presented with mean±SD unless indicated otherwise.

### Example 4

### Geometric Cues Program Melanoma Cells to a Tumorigenic Cancer Stem Cell State

In this example we explored how substrate mechanical properties and geometric cues would influence the tumorigenicity of malignant melanoma cells. The surface chemistry of coverslips (18-mm circular, Fisher Scientific) were modified with 0.5% 3-aminopropyl-triethoxysilane (APTs, 3 min and then washed with distilled water three times) and 0.5% glutaraldehyde (Gluta, 30 min and then washed with distilled water two times) and the coverslips were dried with air. The protocol of preparing polyacrylamide hydrogels was used by employing the mixture of 5% of Acrylamide (AA, Amresco Inc.) and 0.03% of Bis-acrylamide (BAA, Amresco Inc.) for 1 kPa, 10% of AA and 0.1% of BAA for 10 kPA, and 10% of AA and 0.9% of BAA for 100 kPa, 0.1% of ammonium persulfate and Tetramethylethylenediamine were used for the polymerization (Tse, J. R. & Engler, A. J. (2010) supra). 20 μl of the mixtures were pipetted onto the hydrophobic treated glass slides, and the amino-silanized coverslips were added with the treated side down. After appropriate polymerization time, around 25 min, the coverslips chemically attached with the gels were gently detached from the hydrophobic glass slides. Hydrazine hydrate 55% (Fisher Scientific) was employed (2 hour) to modify the substrate surface chemistry, which converts amide groups in polyacrylamide to reactive hydrazide groups (Damljanović, V. et al. (2005) supra). The gels were washed in 5% glacial acetic acid (1 hour, Fluka/Sigma) and in distilled water (1 hour). To generate protein patterns (fibronectin, Sigma), PDMS stamps were fabricated by conventional were fabricated by conventional polymerization methods mixed at a 10:1 (base:hardener) ratio upon a patterned master of photoresist (SU-8, Micro-Chem) created using UV photolithography through a laser printed mask. 25 μg/mL of fibronectin in PBS was incubated with sodium periodate (˜3.5 mg/ml, Sigma-Aldrich) to yield free aldehydes at least 45 min (Lee, J. et al. (2013) supra). The protein solution was applied for 30 min to the top of patterned or unpatterned PDMS, and then dried under air, and transferred to the gel surface. For the protein coated glass, the coverslip treated with APTs and Gluta was used and around 100 μl of the protein solution was pipetted onto the Parafilm (Sigma) and the coverslip was added with the treated side down. Protein coated gels and glass were kept in PBS at fridge before using them in cell culture (see FIG. 3D for schematic of surface modification).

The stiffness of polyacrylamide gels was evaluated by measuring elastic moduli of gels based on atomic force microscopy (AFM) contact force measurements on atomic force microscope (Asylum Research) (Lee, J. et al. (2014) supra). Firstly, the AFM tips (Bruker) were adjusted in air and then in PBS; all force measurements were achieved in PBS. Every stiffness condition was measured with about 10 measurements at different spots. The data was fitted into a Hertz model using IGORPRO software (Wavemetrics). The tip geometry was estimated using a cone architecture to derive the values of Young's modulus.

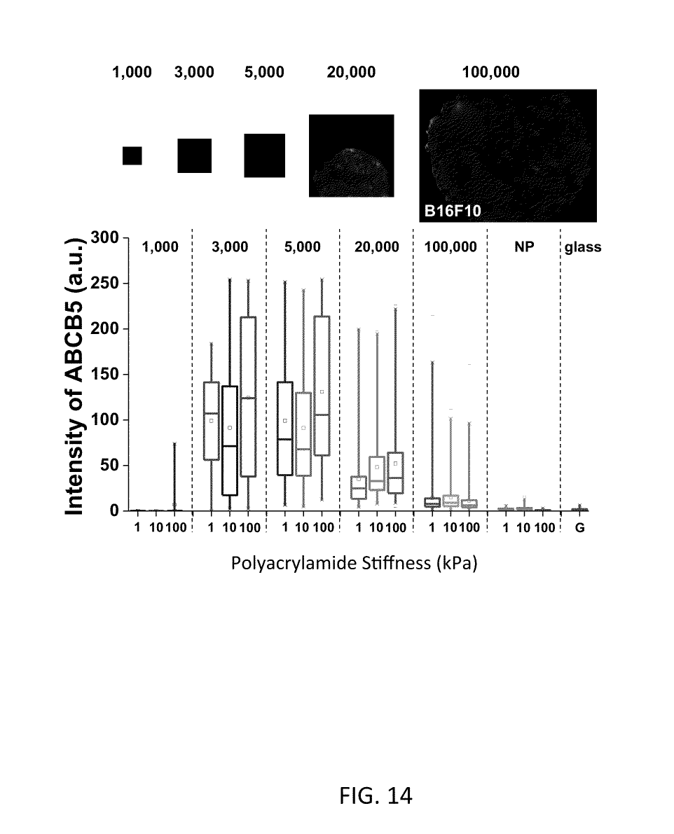

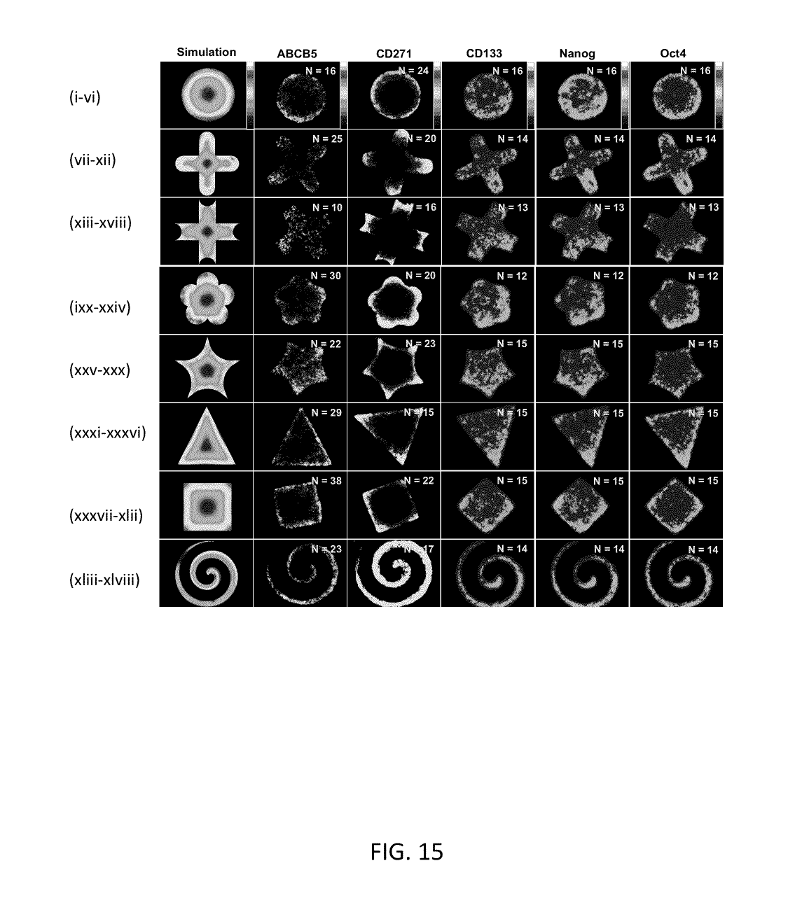

In this study, murine melanoma cell lines, B16F0 (Catalog. No. CRL-6322) and B16F10 (Catalog. No. CRL-6475), were purchased from American Type Culture Collection (ATCC). B16s were thawed from cryopreservation (10% DMSO) and cultured in Dulbecco's Modified Eagle's Medium (DMEM, Corning) high glucose (4.5 g/L) media supplemented with 10% fetal bovine serum (HI FBS, Gibco), and 1% penicillin/streptomycin (p/s, Gibco). Media was changed every 3 days and cells were passaged at nearly 90% confluency using 0.25% Trypsin:EDTA (Gibco). B16s were seeded on patterned and non-patterned surfaces at a cell density at least 10,000 cells/cm² and then the media was changed 1 hour after seeding. Patterns were selected from our modeling of contractile monolayers using ABAQUS software (see FIG. 5B). After incubation for 1, 3, or 5 days, surfaces were fixed with 4% formaldehyde (Ted Pella, Inc.) for 20 min. The fixed cells were permeabilized in 0.1% Triton X-100 in PBS for 30 min and blocked with 1% bovine serum albumin (BSA) for 15 min. Primary antibodies were labeled in 1% BSA in PBS for 2 hours at mom temperature (20° C.) with rabbit anti-ABCB5 (Novusbio NBP 1-77687, 1:500 dilution), CD133 (Mybiosource_MBS462020, 1:500 dilution), or Paxillin (Abcam_ab32084, 1:500 dilution), mouse anti CD271 (Abgent_AM1842a, 1:200 dilution), Nanog (Sigma_N4413, 1:200 dilution), and goat anti Oct4 (Abcam_ab27985, 1:500 dilution). Second antibody labeling was accomplished using the same procedure as BrdU staining with Alexa Fluor 555-conjugated anti-mouse IgG antibody (Life technologies, 1:200 dilution) or donkey anti-Goat IgG (Cy3) antibody (Abcam_ab6949) and Alexa Fluor 647-conjugated anti-mouse IgG antibody (1:200 dilution) along with Alexa Fluor 488-phalloidin (Life technologies, 1:200 dilution) and DAPI (1:5000 dilution) for 20 min in the humid chamber (37° C. with 5% CO₂). Immunofluorescence microscopy was conducted using a Zeiss Axiovert 200 M inverted research-grade microscope (Carl Zeiss, Inc.) or a LSM 700 (Carl Zeiss, Inc.) which is a four laser point scanning confocal with a single pinhole and then immunofluorescent images were analyzed using ImageJ to measure the fluorescence intensity of single cells or ZEN software, respectively. After 5 days in culture B16 cells began to express the putative melanoma stem cell markers ABCB5, CD133 and CD271. The expression of these markers was found to depend on pattern size where an optimal area range led to the highest fraction of cells with robust expression (3000 μm²-20,000 μm²) with a slight dependence on hydrogel stiffness (FIG. 14). We overlaid multiple immunofluorescence images of populations of cells adherent to 20,000 μm² shapes in Image J (NIH) to generate heat maps. FIG. 15 shows heat maps for our tested geometries where regions of curvature and pinch points at the perimeter induced high expression of cancer stem cell markers.

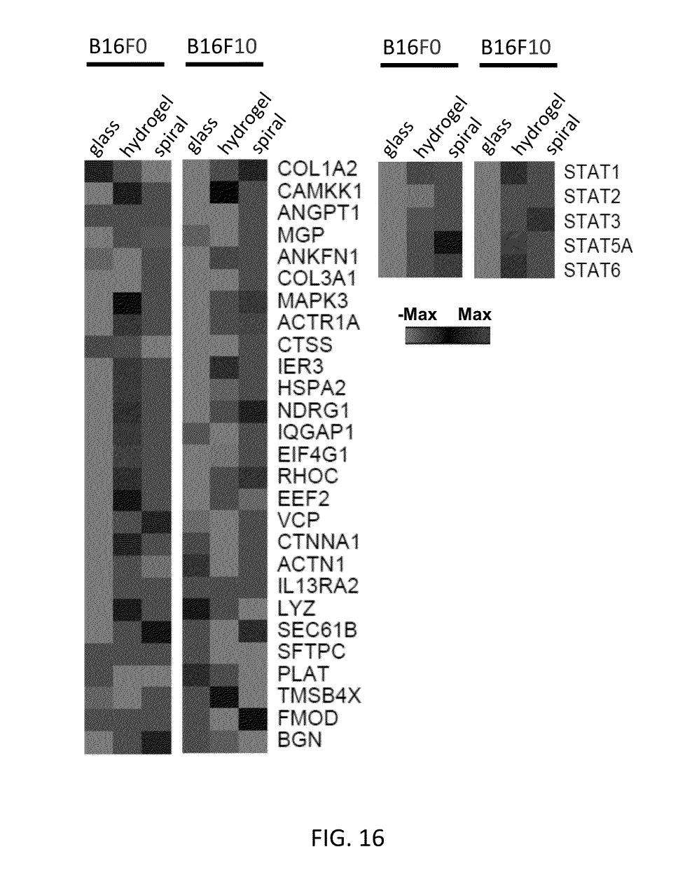

Next we selected the 20,000 μm² spiral shape that showed the highest expression of stem cell markers for RNA isolation and microarray analysis. TRIZOL reagent (Invitrogen) was used for lysing adherent cells and purified according to vendor's protocol. High quality samples containing nearly 1 μg of total RNA at concentration around 100 ng/μl were sent to the University of Illinois Roy J. Carver Biotechnology Center for labeling and hybridization on Illumina bead arrays. The procedures for reverse transcription, labeling and hybridization were performed at the functional Genomics facility using standard Illumina's protocols. Illumina gene array data was preprocessed using GenePattern. The background values were subtracted, thresholded and filtered for specified fold changes. The data was then normalized using the quantile method. Heat maps of fold changes in gene expression were visualized using the Gene-E software package. Possible genetic pathways were analyzed with DAVID Knowledgebase (http://david.abcc.ncifcrf.gov/). Gene expression analysis revealed increased expression of transcripts associated with metastasis and Jak-STAT signaling pathways (FIG. 16). In addition there was an increase in mitogen activated protein kinase (MAPK) signaling, particularly the p53 MAPK activity.

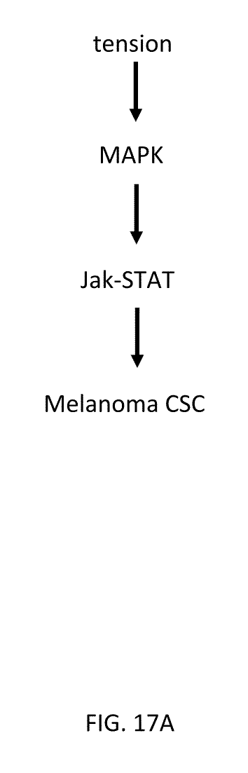

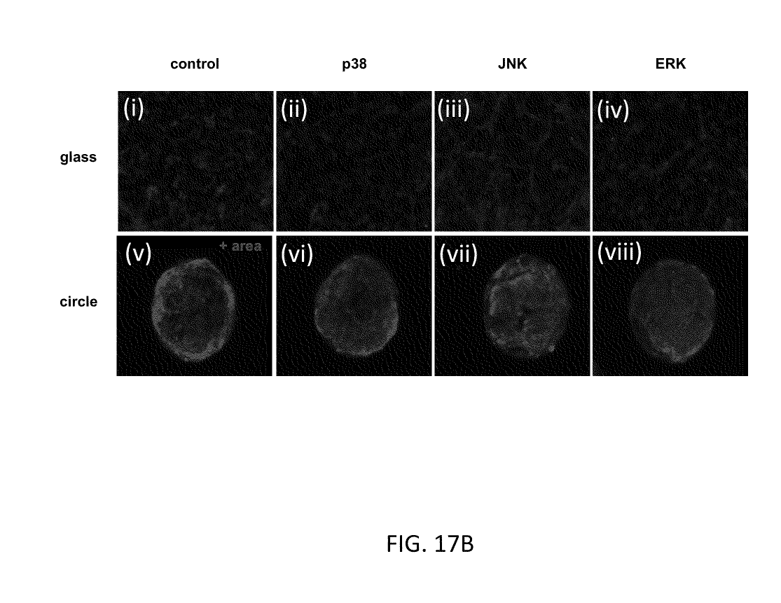

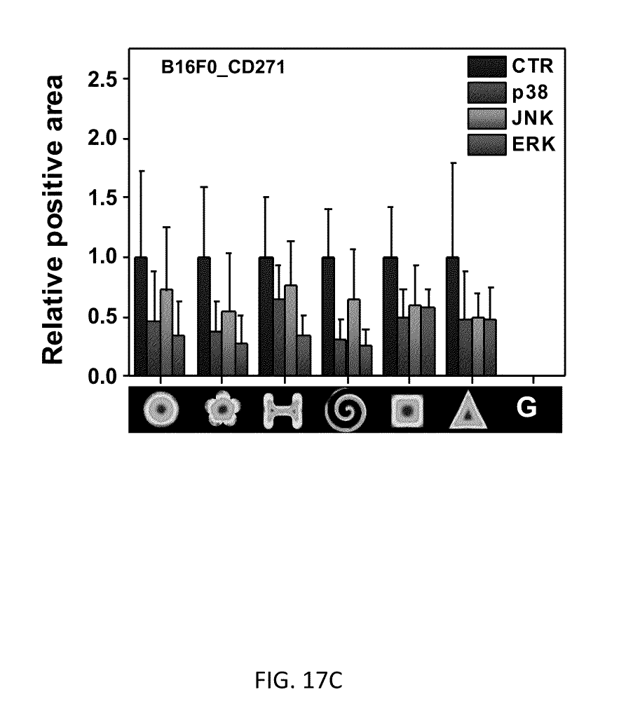

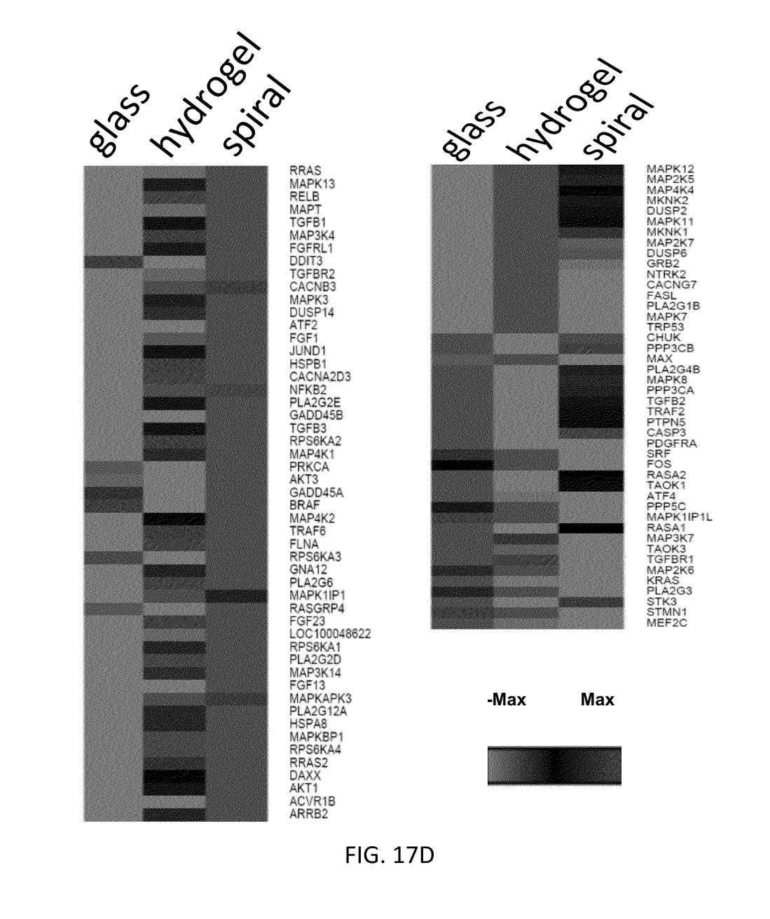

To test the role of MAPK signaling during melanoma programming, we selected MAP kinase inhibitors, SB202190 (p38), SP600125 (JNK), and FR180204 (ERK1/2) (Calbiochem), and supplemented the media of the following molecules at 6 μM after cell seeding and with each media change (Kilian, K A., Bugarija, B., Lahn, B. T. & Mrksich, M. “Geometric cues for directing the differentiation of mesenchymal stem cells,” Proc. Natl. Acad. Sci., U.S.A. 107:4872-4877 (2010)). FIG. 17A shows a strategy for how adding inhibitors can lead to a decrease in the perimeter expression of cancer stem cell markers.

At least, three repeats were done for each experimental group; the number of each cell measured was at least 50 cells per each condition and multi unless indicated otherwise. The intensity value for each cell was obtained from cytoplasmic staining intensity minus backgrounds for each condition (FIG. 17B). Statistical significance was determined via Student's t test, where p<0.05. The data is presented with mean+SD unless indicated otherwise. For producing immunofluorescent heat maps (FIG. 17C, D), B16s (same passage) cultured on desired patterns with monolayers were imaged on the same conditions (staining and imaging day and microscope setting). Raw fluorescent images were aligned in image J with the same positioning, incorporated into a Z stack and the mean intensity calculated for heat map generation (FIG. 17D).

### REFERENCES

All patents, patent applications, patent application publications and other publications cited herein are hereby incorporated by reference as if set forth in their entirety.

It should be understood that the methods, procedures, operations, composition, and systems illustrated in figures may be modified without departing from the spirit of the present disclosure. For example, these methods, procedures, operations, devices and systems may comprise more or fewer steps or components than appear herein, and these steps or components may be combined with one another, in part or in whole.

Furthermore, the present disclosure is not to be limited in terms of the particular embodiments described in this application, which are intended as illustrations of various embodiments. Many modifications and variations can be made without departing from its scope and spirit. Functionally equivalent methods and apparatuses within the scope of the disclosure, in addition to those enumerated herein, will be apparent to those skilled in the art from the foregoing descriptions.

The invention claimed is:

1. A composition for programming a cell, comprising:
a substrate; and
a cell adhesion agent,
wherein the substrate comprises a surface having a micropatterned object and the cell adhesion agent is immobilized within a first area defined by the micropatterned object.

2. The composition of claim 1, wherein the substrate comprises:
a first layer, wherein the first layer comprises the surface;
a second layer, wherein the second layer comprises a first chemistry coupled to the first layer, the second layer provides the first area defined by the micropatterned object; and
a third layer, wherein the third layer provides a second chemistry coupled to the first layer, the third layer provides a second area defined by the micropatterned object.

3. The composition of claim 2, wherein the cell adhesion agent is immobilized within the first area by coupling to the first chemistry of the second layer.

4. The composition of claim 2, wherein the first chemistry comprises an alkanethiol compound and the second chemistry comprises a triethylene glycol-alkanethiol.

5. The composition of claim 1, wherein the substrate comprises glass.

6. The composition of claim 1, wherein the surface comprises a Au layer or a hydrogel layer.

7. The composition of claim 1, wherein the cell adhesion agent is a protein.

8. The composition of claim 7, wherein the protein is selected from a group consisting of fibronectin, laminin, vitronectin and collagen, or mixtures thereof.

9. The composition of claim 1, wherein the cell is a somatic cell.

10. The composition of claim 1, wherein the micropatterned object comprises a geometric shape.

11. The composition of claim 1, wherein the substrate is a hydrogel having a stiffness in the range from about 0.1 kPa to about 1 MPa.

12. A method for programming a cell, comprising:
providing a composition, comprising:
a substrate; and
a cell adhesion agent;
wherein the substrate comprises a surface having a micropatterned object and the cell adhesion agent is immobilized within a first area defined by the micropatterned object.
contacting a cell with the composition to form an adherent cell culture; and
culturing the adherent cell culture for a period to effect programming of the cell.

13. The method of claim 12, wherein the adherent cell culture comprises the cell contacting the cell adhesion agent.

14. The method of claim 12, wherein the substrate comprises:
a first layer comprising an Au layer, wherein the first layer comprises the surface;
a second layer, wherein the second layer comprises an alkanethiol compound coupled to the first layer, the second layer provides the first area defined by the micropatterned object; and
a third layer, wherein the third layer comprises a triethylene glycol-alkanethiol compound coupled to the first layer, the third layer provides a second area defined by the micropatterned object.

15. The method of claim 14, wherein the micropatterned object comprises a geometric shape.

16. The method of claim 12, wherein the substrate comprises:
a first layer comprising an acrylamide hydrogel, wherein the first layer comprises the surface;
a second layer, wherein the second layer comprises an oxidized form of the cell adhesion agent coupled to the first layer, the second layer provides the first area defined by the micropatterned object; and
a third layer, wherein the third layer comprises a hydrazine compound coupled to the first layer, the third layer provides a second area defined by the micropatterned object.

17. The method of claim 16, wherein the micropatterned object comprises a geometric shape.

18. The method of claim 16, wherein the hydrogel comprises a stiffness in the range from about 0.1 kPa to about 1 MPa.

19. The method of claim 12, further comprising transforming the adherent cell culture.

20. A kit for programming a cell, comprising:
a premade composition for programming a cell, comprising:
a substrate; and
a cell adhesion agent,
wherein the substrate comprises a surface having a micropatterned object and the cell adhesion agent is immobilized within a first area defined by the micropatterned object.
